(19) United States
(12) Patent Application Publication
(10) Pub. No.: US 2026/0191432 A1
(43) Pub. Date: Jul. 9, 2026

(54) METHODS AND SYSTEMS FOR MONITORING AND TREATING DISORDERS WITH CORTISOL MEASUREMENTS

(71) Applicant: Adaptyx Biosciences, Inc., Half Moon Bay, CA (US)

(72) Inventors: Alexander Yoshikawa, Half Moon Bay, CA (US); Ian Thompson, Half Moon Bay, CA (US); Vijit Sabnis, Half Moon Bay, CA (US); Elena Christofides, Half Moon Bay, CA (US); Vijaykumar Rajasekhar, Half Moon Bay, CA (US); Daniel Grossman, Half Moon Bay, CA (US)

(73) Assignee: Adaptyx Biosciences, Inc., Half Moon Bay, CA (US)

(21) Appl. No.: 19/441,440

(22) Filed: Jan. 6, 2026

Related U.S. Application Data
(60) Provisional application No. 63/742,400, filed on Jan. 6, 2025.

(51) Int. Cl. A61B 5/145; A61B 5/00; A61B 5/1473; A61B 5/1495

(52) U.S. Cl. CPC A61B 5/14514; A61B 5/0017; A61B 5/14546; A61B 5/14735; A61B 5/4839; A61B 5/685; A61B 5/7203; A61B 5/7282; A61B 5/1495; A61B 2562/0271

(57) ABSTRACT

The method can include: determining a set of analyte levels, and extracting a set of features from the set of analyte levels. The system can include: a set of analyte binding probes, a reader (e.g., an electrochemical reader, optoelectronic reader, etc.), and a processing system. In variants, the system and/or method can function to monitor cortisol levels and/or levels of one or more other analytes.

### CROSS-REFERENCE TO RELATED APPLICATIONS

This application claims the benefit of U.S. Provisional Application No. 63/742,400 filed 6 Jan. 2025, which is incorporated in its entirety by this reference.

### TECHNICAL FIELD

This invention relates generally to the biosensing field, and more specifically to a new and useful system and method in the biosensing field.

### BACKGROUND OF THE INVENTION

Cortisol is a regulating hormone of the stress response, and thus has wide-reaching effects on physiological processes. However, measuring and monitoring cortisol can be challenging, especially in treating and diagnosing diseases in which cortisol has become dysregulated.

### BRIEF DESCRIPTION OF THE SEVERAL VIEWS OF THE DRAWINGS

FIG. 1: A schematic representation of a variant of the method.

FIG. 2: A schematic representation of a variant of the system.

FIG. 3: A schematic representation of an example of the method.

FIG. 4: A schematic representation of an example of the method, including determining a health metric based on a set of features for at least two analytes.

FIG. 5: A schematic representation of an example of the method, including determining an analyte level by processing signals from at least two analyte binding probes.

FIG. 6: The HPA axis. Overview of the HPA axis and mechanism of cortisol regulation and feedback.

FIG. 7: Features of the diurnal cortisol cycle. A) Typical diurnal cycle of cortisol throughout a 24 hour period. Features such as maximum value, minimum value, peak-to-trough height, peak-to-trough time, CAR (cortisol awakening response), and slopes of changes can be measured. B) Shows additional features of a cortisol profile that can be measured.

FIG. 8: Cortisol profile of a Cushing patient. Example of the measured cortisol profile of a Cushing patient (red line) versus healthy subjects (blue is the mean of 214 healthy subjects).

FIG. 9: Real-time cortisol sensing with a benchtop device. Samples of known or unknown concentrations of cortisol are connected to a programmable motorized valve via fluidic tubing. The outlet of the valve is connected to the inlet of a microfluidic sample chamber using fluidic tubing. Binding probes such as aptamer switches are conjugated to magnetic particles, which are immobilized in the chamber using a permanent magnet adhered to the top of the microfluidic chip. The outlet of the microfluidic chip is attached to a syringe with a blunt-tip needle on a programmable syringe pump using fluidic tubing. The syringe pump drives the fluid through the system from the 5 mL samples all the way to the syringe, which acts as a waste reservoir. The microfluidic chip is affixed to the stage of an inverted fluorescence microscope. The microscope delivers light from an LED light engine to excite luminescent molecules within the binding probes. The emission light of the luminescent molecules is imaged by a camera attached to the microscope. The images can be used to quantify the signal of the binding probes over time, which in some embodiments are mapped to concentrations using a calibration curve.

FIG. 10: Real-time cortisol measurement data in-vitro. Data from benchtop setup demonstrating real-time cortisol sensing over physiologically relevant concentrations. Samples of 0, 10, 50, and 100 nM cortisol solution were run through a benchtop system in a particular sequence, holding each condition for 40 minutes. The images from the benchtop system were analyzed and calibrated to generate a curve representing the measured concentration over time (blue curve). The actual concentration step function representing the concentration sequence is also shown (grey curve).

FIG. 11: Example of body-worn device. (A) In some embodiments, a skin interfacing device allows access to interstitial fluid (ISF), and is connected to an optical detector. The skin interfacing device penetrates the dermis, and contains analyte binding probes which interact with analytes within ISF. Analyte binding probes undergo a change in signal upon interacting with the analyte, and these changes are detected by the optical detector. In some embodiments, the analyte is a cortisol and/or other reference molecules. (B) In some embodiments, the analyte binding probe is a molecule that interacts with the analyte through reversible target binding, and undergoes a binding-induced conformational change in response to this binding. In some embodiments, this analyte binding probe contains optical reporters (fluorophore and quencher) that produce a change in signal in response to this conformational change, and this signal change can be measured by the optical detector. (C) In some embodiments, the device is applied to a patient's skin, and measures an analyte concentration within the ISF, then stores and/or transmits this data to an external source. The data on this external source is then used by healthcare providers to view analyte levels, and make clinical decisions based on values, trends, alarms, or fitting to an expected analyte concentration profile over time. In some embodiments, the analyte is cortisol, and the device is used to assist in diagnosing, monitoring, and treating disorders or conditions impacted by cortisol rhythm.

FIG. 12: A specific example of in vivo monitoring of cortisol levels using the system, after a dose of synthetic cortisol (e.g., hydrocortisone).

FIG. 13: An illustrative example of a normal cortisol curve.

FIG. 14: An illustrative example of a cortisol curve showing hypercortisolism.

FIG. 15: An illustrative example of a cortisol curve showing hypercortisolism and a low-dose dexamethasone suppression test.

FIG. 16: An illustrative example of a cortisol curve showing hypercortisolism with therapeutic intervention.

FIG. 17: An illustrative example of a cortisol curve showing hypercortisolism treated with hydrocortisone.

### DETAILED DESCRIPTION OF THE INVENTION

The following description of the embodiments of the invention is not intended to limit the invention to these embodiments, but rather to enable any person skilled in the art to make and use this invention.

### 1. Overview

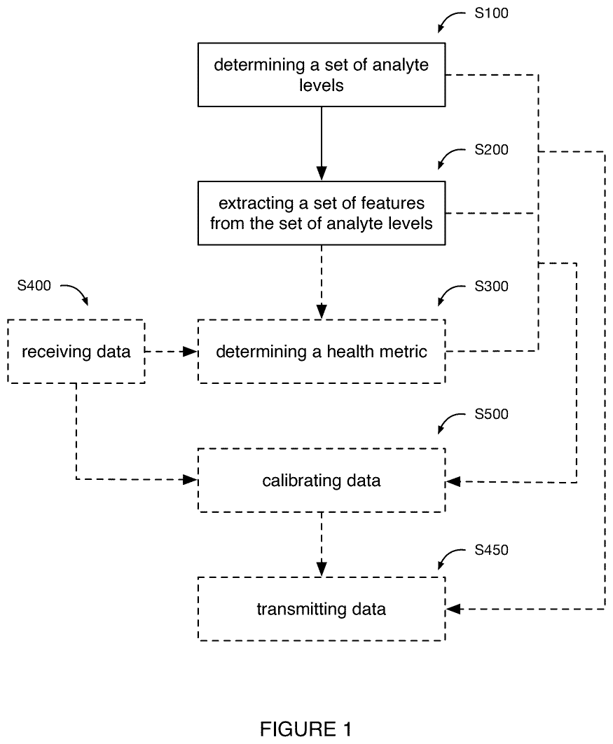

As shown in FIG. 1, the method can include: determining a set of analyte levels S100, and extracting a set of features from the set of analyte levels S200. The method can optionally include determining a health metric S300, receiving data S400, transmitting data S450, and/or any other suitable steps. However, the method can be otherwise performed.

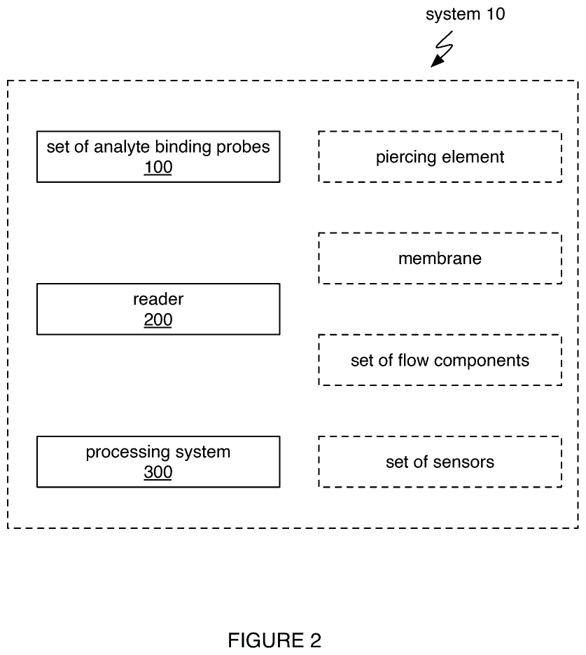

As shown in FIG. 2, the system can include: a set of analyte binding probes, a reader (e.g., an electrochemical and/or optoelectronic reader), and a processing system. The system can optionally include one or more of: a piercing element, a membrane, a set of flow components (e.g., a flow cell assembly, a pump, an input reservoir, an output reservoir, etc.), and/or any other suitable components. However, the system can be otherwise configured.

In variants, the system and/or method can function to monitor cortisol levels and/or levels of one or more other analytes. In variants, the system and/or method can function to aid in the diagnosis, monitoring, and/or treatment of a condition and/or other health parameter (e.g., pain, inflammation, cognitive function, stress, athletic recovery, surgery recovery, fertility, metabolic health, menstruation, diurnal cortisol rhythm, etc.). Specific examples of conditions include: adrenal insufficiency, Cushing disease, Cushing syndrome, diabetes, hypertension, obesity, osteoporosis, mild autonomous cortisol secretion (MACS), hypercortisolemia, a sleep disorder, a mental disorder, long COVID, and/or any other condition associated with cortisol. In variants, the system and/or method can function to adjust the administration of a drug (e.g., titrate drug dosage, adjust dosing timing, etc.) to achieve a target (e.g., a target cortisol level, a target cortisol rhythm, etc.).

### 2. Technical Advantages

Variants of the technology can confer one or more advantages over conventional technologies.

Cortisol is a regulator of numerous physiologic processes and is a regulating hormone of the stress response, and thus has wide-reaching effects on physiological processes including metabolism, immunity, mental health, and circadian rhythm. While healthy cortisol levels follow a characteristic diurnal rhythm or circadian rhythm, a variety of adrenal diseases and other conditions are characterized by cortisol levels that are either too low, too high, or dysrhythmic. Current technologies that measure cortisol can only measure cortisol at a single point in time. These current diagnostic tests are inconvenient and sometimes inaccurate when used for the diagnosis and treatment of endocrine disorders as they cannot capture cortisol's circadian variability. These tests are even less likely to capture cortisol's ultradian rhythm, pulses of cortisol every 60-120 minutes thought to be responsible for daytime wakefulness and, with a relative absence at night, nighttime sleep consolidation or, with nighttime aberrancy, insomnia. Therefore, there is a clear need for improved methods for measuring cortisol for diagnosing, monitoring, and treating the various disorders or health conditions in which cortisol plays a role. In variants, this invention describes methods of leveraging periodic cortisol measurements from a device worn on the body of a subject to measure cortisol, to assist in diagnosing, monitoring, and/or treating disorders or conditions impacted by cortisol rhythm.

Cortisol is a key regulating hormone of the stress response, and thus has wide-reaching effects on physiological processes including metabolism, immunity, mental health, and circadian rhythm. While healthy cortisol levels follow a characteristic diurnal rhythm, a variety of adrenal diseases are characterized by cortisol levels that are either too low, too high, or lacking in the healthy physiological rhythm.

Continuous measurement of cortisol would revolutionize both the diagnosis and management of Cushing disease (prevalence of ~40 per million in the US) and Mild Autonomous Cortisol Secretion (MACS; estimated prevalence of >2% in adult population), where elevated cortisol levels negatively impact patient mortality and quality of life. Current single time point diagnostic tests for these conditions are inconvenient and sometimes inaccurate as they cannot capture cortisol's diurnal variability. Furthermore, since cortisol fluctuations can occur over seconds to minutes, single time point testing may miss these shorter time scale fluctuations.

Pharmaceutical interventions are available to lower cortisol levels, but dosing these drugs without the ability to track the resulting changes in patient cortisol levels poses a substantial risk of excessive reduction in cortisol production which can lead to adrenal failure and death. Cortisol monitoring could also transform the dosing of long-term oral glucocorticoid drugs, which are prescribed to 1.2% of the total population and lead to the onset of adrenal insufficiency in approximately half of this group, thus requiring careful tapering to return patients to healthy cortisol levels. Taken together, continuous cortisol monitoring for adrenal health represents an opportunity to improve the treatment of millions of patients in the US, which currently comes at a substantial cost to the healthcare system, with ~$1B of expenditure on Cushing disease pharmaceuticals alone.

However, further advantages can be provided by the system and method disclosed herein.

### 3. Method

As shown in FIG. 1, the method can include: determining a set of analyte levels S100, and extracting a set of features from the set of analyte levels S200. The method can optionally include determining a health metric S300, receiving data S400, transmitting data S450, calibrating data S500, and/or any other suitable steps. All or portions of the method can be performed automatically, manually, semi-automatically, and/or otherwise performed.

Variants of the method can use systems and/or methods as described in U.S. application Ser. No. 18/813,369 filed 23 Aug. 2024, International PCT Application No. PCT/US2025/010333 filed 3 Jan. 2025, U.S. application Ser. No. 18/981,409 filed 13 Dec. 2024, U.S. application Ser. No. 19/098,929 filed 2 Apr. 2025, each of which is incorporated in its entirety by this reference.

In variants, the system and/or method can function to measure analyte level(s) in a sample of a user (e.g., subject, patient, etc.). The user is preferably a human user, but can alternatively be another animal, another biological system, a nonbiological system (e.g., for testing, for industrial process measurements, etc.), and/or any other subject. The sample can be a biological sample or a nonbiological sample. Examples of biological samples include: interstitial fluid (e.g., dermal interstitial fluid), dermal interstitial fluid, extracellular fluid, intracellular fluid, blood, serum, plasma, saliva, sweat, urine, cerebrospinal fluid (CSF), an organ (e.g., dermis, brain, kidney, liver, eye, muscle, etc.), any biofluid, and/or any other biological sample. The sample can optionally be a fluid or contain a fluid. As used herein, an analyte level can be a concentration, a metric indicative of concentration, and/or any other quantification of the analyte. Examples of analytes include: cortisol, glucose, cholesterol, lactate, potassium, sodium, adrenocorticotropic hormone (ACTH), corticotropin-releasing hormone (CRH), methylprednisolone, insulin, glucagon, DHEA (dehydroepiandrosterone), DHEA-S (dehydroepiandrosterone sulfate), inflammation markers, a glucocorticoid (e.g., prednisone, pregnisolone, etc.), cortisone, corticosterone, corticosteroid (e.g., corticosteroid 11-beta-dehydrogenase isozyme 2), estrogen, progesterone, estradiol, testosterone, follicle stimulating hormone (FSH), luteinizing hormone (LH), a cortisol metabolite (e.g., alpha-THF, beta-THF), a cortisone metabolite (e.g., beta-THE), a drug (e.g., a cortisol-modulating drug), and/or any other biological analyte. Examples of drugs (e.g., medications) include: dexamethasone, adrenal steroidogenesis inhibitor, adrenal steroidogenesis inhibitor, glucocorticoid, glucocorticoid receptor blocker, and/or any other molecule administered to a user.

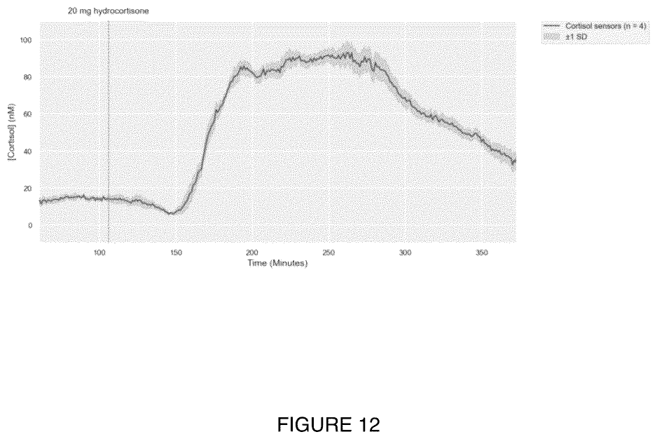

Determining a set of analyte levels S100 functions to monitor one or more analytes over time. Analyte level(s) can optionally be measured continuously, intermittently, iteratively, periodically, in real time, in response to a request, and/or at any other time. The set of analyte levels can optionally be or include a time series of analyte levels (e.g., time series of cortisol levels). An example is shown in FIG. 12. In an example, an analyte level (e.g., cortisol level) can be measured periodically. In examples, the time series of analyte levels can include an analyte level at least every 24 hours, at least every hour, at least every 30 min, at least every 10 min, at least every 5 min, at least every 2 min, at least every min, and/or at least every 30 sec. The time series of analyte levels can correspond to a time period (e.g., where the time period spans the first and last analyte level in the time series). In specific examples, the time period can be: at least 1 hour, at least 2 hours, at least 5 hours, at least 6 hours, at least 10 hours, at least 12 hours, at least 24 hours, at least 48 hours, at least 72 hours, and/or at least 1 week.

Determining each analyte level in the set of analyte levels (e.g., each analyte level in the time series of analyte levels) can optionally include: measuring a set of signals from a set of analyte binding probes, and determining an analyte level based on the set of signals. The set of analyte binding probes can be in contact with all or a portion of the sample (e.g., interstitial fluid) and/or all or a portion of analytes from the sample. In a specific example, interstitial fluid is filtered through a membrane to create dialysate, and the set of analyte binding probes contact the dialysate. The set of signals can be measured using the reader (e.g., an electrochemical or optoelectronic reader). The signals can be light, electric, and/or any other type of signal. The signal may be analog or digital. In a specific example, for a light signal, the reader can measure the wavelength, power, intensity, pulse counts (e.g., pulse counts for a specific wavelength, pulse counts for a specific wavelength band, etc.), and/or any other light parameters. In an example, determining each analyte level can include: using the reader, recording a signal emitted by a set of analyte binding probes, and determining the cortisol level based on the recorded signal. In a specific example, an analyte binding probe can include an aptamer (e.g., an aptamer switch) configured to bind to the analyte (e.g., cortisol) and configured to change in conformation in response to binding to the analyte. In a specific example, the analyte binding probe can include a reporter molecule (e.g., aptamer is bound to the reporter molecule directly or indirectly, via a linker), wherein the reporter molecule emits a signal in response to the change in conformation of the aptamer. In a first example, the reporter molecules can include a redox reporter. In a second example the reporter molecules can include a fluorophore and/or quencher.

Determining an analyte level based on the set of signals can include mapping the set of signals and/or feature thereof (e.g., pulse count) to an analyte level (e.g., concentration). In a specific example, a calibration factor can be used to transform the set of signals to analyte levels.

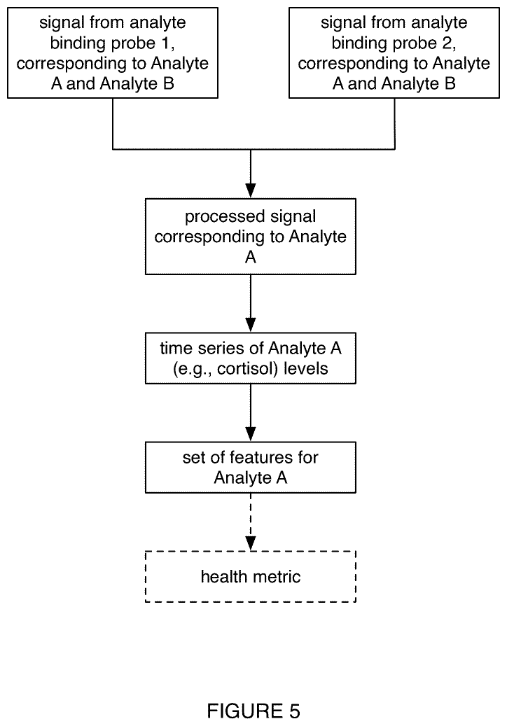

In a first variant, a set of signals from a set of analyte binding probes are used to determine the analyte level (e.g., cortisol level), where each aptamer in the set of analyte binding probes has the same nucleotide sequence. In a second variant, a first set of signals from a first set of analyte binding probes and a second set of signals from a second set of analyte binding probes are used to determine the analyte level (e.g., cortisol level), where aptamers in the first set of analyte binding probes have a different sequence than aptamers in the second set of analyte binding probes. For example, the aptamer in the first set of analyte binding probes can be configured to bind to the analyte (e.g., cortisol) with a first binding affinity and configured to bind to a secondary analyte (e.g., non-cortisol molecule) with a second binding affinity, and the aptamer in the second set of analyte binding probes can be configured to bind to the analyte (e.g., cortisol) with a third binding affinity and configured to bind to the secondary analyte with a fourth binding affinity. In variants, if the specificity of a single analyte-binding aptamer is below a threshold, two or more different aptamers with varying specificities to other analytes (e.g., other steroidal molecules) can be used to determine the analyte level. The number of different sets of analyte binding probes (with different aptamers) can be: 1, 2, 3, 4, 5, or more than 5. An example is shown in FIG. 5.

Analyte levels can be determined for a single analyte or for multiple analytes (e.g., at least 2, at least 3, at least 4, etc.). In a first embodiment, testosterone, aldosterone, and/or cortisol are monitored (e.g., cortisol and aldosterone; cortisol and testosterone; cortisol, testosterone, and aldosterone). In a second embodiment, a cortisol-modulating drug and cortisol are monitored (e.g., for drug titration). In a third embodiment, cortisol, dexamethasone, and potassium and/or sodium are monitored (e.g., for congenital adrenal hyperplasia). In a fourth embodiment, cortisol, glucose, lactate, and/or insulin, are monitored. In a specific example, this can be used for hypercortisolemia and/or diabetic users to monitor how cortisol affects the users' metabolic pathway. In a fifth embodiment, cortisol and dexamethasone are monitored. In a first specific example, monitoring cortisol and dexamethasone can be used to verify if a user has taken dexamethasone (e.g., the health metric, determined via S300, can include a confirmation that the user has taken dexamethasone). In a second specific example, monitoring cortisol and dexamethasone can be used to determine a DST score (e.g., the health metric, determined via S300, can include a dexamethasone suppression test score). In a third specific example, monitoring cortisol and dexamethasone can be used to titrate the amount of dexamethasone the user is taking. In a fourth specific example, monitoring cortisol and dexamethasone can be used to characterize how a patient is metabolizing dexamethasone (e.g., the health metric, determined via S300, can include a characterization of dexamethasone metabolism of the user). In a fifth specific example, monitoring cortisol and dexamethasone can be used to characterize how dexamethasone is suppressing cortisol in the user (e.g., the health metric, determined via S300, can include a characterization of dexamethasone suppression of cortisol). In a sixth embodiment, cortisol and DHEA-S are monitored. In a seventh embodiment, cortisol and potassium are monitored. In an eighth embodiment, cortisol, potassium, and sodium are monitored.

The signals and/or analyte levels can optionally be processed. Examples of processing include: correcting (e.g., correcting for binding kinetics, correcting for temperature, etc.), calibrating, filtering, smoothing, normalizing, transforming, dimensionality reduction, statistical analysis, downsampling, upsampling, fitting, denoising, a combination thereof, and/or any other processing methods. In a specific example, the method can include: determining that a set of signals and/or corresponding set of analyte levels corresponds to a cortisol awakening response (CAR) (e.g., based on the signals and/or analyte levels, based on the rate of change, based on a predetermined time window, etc.), and correcting the set of signals and/or corresponding set of analyte levels for binding kinetics (e.g., a low on-rate relative to the CAR time).

However, one or more analyte levels can be otherwise determined.

Extracting a set of features from the set of analyte levels S200 functions to extract relevant information from one or more sets of analyte levels (e.g., one or more time series of analyte levels).

The features can be semantic or nonsemantic features. Examples of features include: a mean, a peak, a trough (e.g., nadir), an integral (e.g., area under the curve), a rate of change, a curve shape, a time at which a feature occurs (e.g., a time at which an analyte level increase occurs), comparisons (e.g., differences, ratios, etc.) thereof, changes thereof, and/or any other features. Features can correspond to the entire set of analyte levels and/or a time window within the set of analyte levels (e.g., 24 hrs, 12 hrs, a day, etc.). In examples, the set of features can include one or more of: daily maximum (e.g., daily peak), daily minimum (e . . . g, daily trough), a time at which the daily minimum occurs, time at which the daily maximum occurs, mean analyte level throughout a 24 hour period, mean analyte level throughout a period longer than 1 hour but shorter than 24 hours, mean analyte level throughout a period longer than 5 minutes but shorter than 1 hour, mean analyte through a period longer than 24 hours but shorter than or equal to 2 weeks, maximum analyte level during a 24 hour period, minimum analyte level during a 24 hour period, difference between a daily maximum and daily minimum analyte measurement, the number of local analyte maxima above a certain threshold from the surrounding local minima (e.g., cortisol spikes) occurring within a 24 hour period, maximum analyte level occurring within 2-hours after a meal, time between a daily maximum and daily minimum, and/or any other feature corresponding to a time window. In an example, a feature can be a start time of an increase in cortisol secretion. In a specific example, the start time of an increase in cortisol secretion is marked by an increase in the rate of change above a certain threshold after a certain time of day. In a specific example, the start time of the increase in cortisol secretion is marked by the cortisol level crossing a threshold at a defined percentage of the daily maximum cortisol peak. In another specific example, the start time of an increase in cortisol secretion is marked by the cortisol level crossing a threshold at a defined percentage of the difference between daily maximum cortisol peak and daily minimum cortisol trough. In another example, a feature can be the cortisol awakening response (CAR). In another example, a feature can be the cortisol response within 1 hour of waking. In another example, a feature can be the morning cortisol level after administration of a 1 mg dose of dexamethasone before bedtime. In another example, a feature can be the difference between the morning cortisol level on a day without dexamethasone administration and the morning cortisol level after a 1 mg dose of dexamethasone before bedtime. In another example, a feature can be an ultradian rhythm of cortisol.

Features can be determined using a model (e.g., classical or traditional model, machine learning model, etc.), fourier analysis, dimensionality reduction (e.g., PCA, t-SNE, LDA, etc.), processing methods, and/or any other feature extraction. Examples of processing include: correcting (e.g., correcting for binding kinetics, correcting for temperature, etc.), calibrating, filtering (e.g., low-pass filtering), smoothing, normalizing, transforming, dimensionality reduction, statistical analysis, downsampling, upsampling, fitting, denoising, comparison methods (e.g., matching, distance metrics, thresholds, etc.), a combination thereof, and/or any other processing methods.

However, any other features can be determined based on the set of analyte levels.

The method can optionally include determining a health metric S300, which functions to interpret and/or analyze the set of features. The health metric can be qualitative, quantitative, relative, discrete, continuous, a classification, numeric, binary, and/or be otherwise characterized.

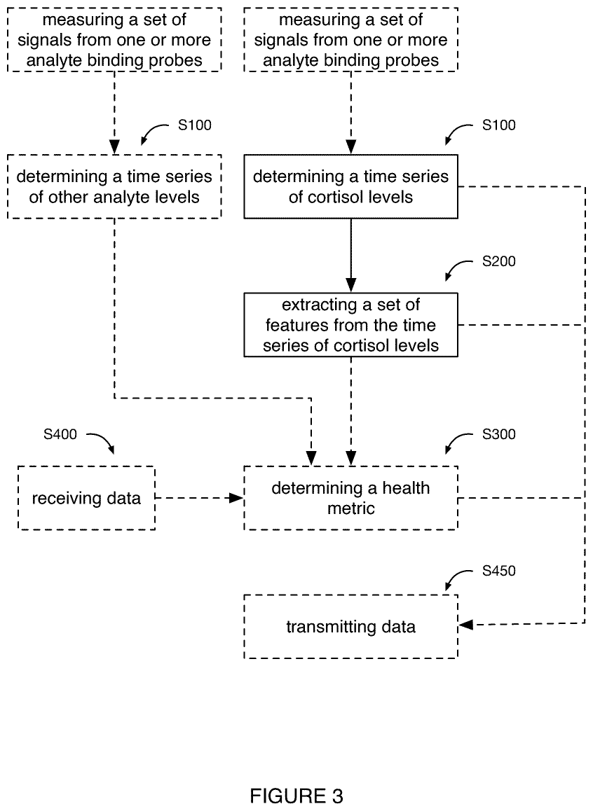

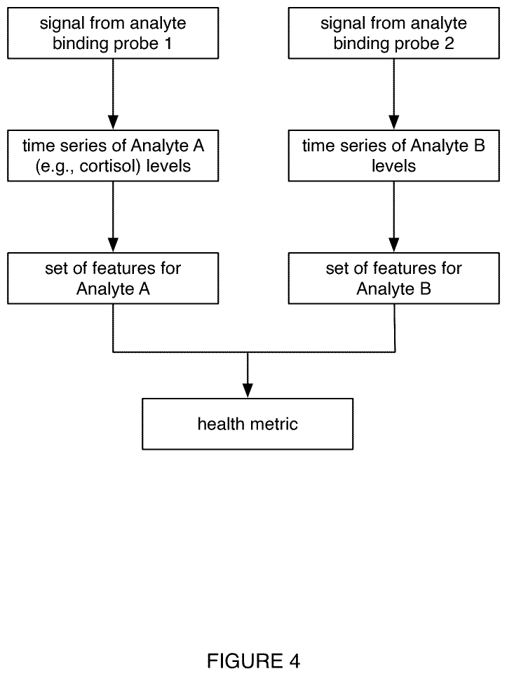

The health metric can be determined based on: a set of analyte levels for one or more analytes, a set of features for one or more sets of analyte levels, data (e.g., biomarker data, environmental data, patient reported data, any data received via S400, etc.), and/or any other suitable information. In a first example, the health metric can be determined based on a set of features extracted from a time series of analyte levels (e.g., a time series of cortisol levels). In a second example, the health metric can be determined based on a set of features extracted from a time series of analyte levels for a first analyte (e.g., cortisol) and a set of features extracted from a time series of analyte levels for a second analyte. Examples are shown in FIG. 3 and FIG. 4.

The health metric can be or include: a diagnosis of a condition, a characterization of a user state, a characterization of an analyte level (e.g., characterization of a specific analyte level, characterization of an analyte change over time, characterization of an analyte rhythm such as a diurnal cortisol rhythm, etc.), a characterization (e.g., analysis) of a condition, a characterization of an effect of a first analyte on a second analyte, a score, a confirmation that a user has taken a drug, a characterization of an analyte (e.g., drug) metabolism by the user, a characterization of analyte modulation by a user action, a characterization of analyte modulation by a drug (e.g., dexamethasone suppression of cortisol), a comparison to a cohort (e.g., comparison of analyte levels to a cohort), a recommended action, a drug administration adjustment, diagnostic intervention analyses (e.g., Low-Dose Dexamethasone-Suppression Test, High-Dose Dexamethasone-Suppression Test, etc.), and/or any other metric. The user state (e.g., a health state, a mental state, etc.) can include: pain, inflammation, cognitive function (e.g., focus level, decision making, brain fog, etc.), stress, athletic recovery, surgery recovery, gender, fertility, metabolism and/or metabolic health, menstruation, pregnancy, and/or any other parameter of the user.

Examples of conditions include: adrenal insufficiency, adrenal tumors, Cushing disease, Cushing syndrome, Cyclical Cushing syndrome, diabetes, hypertension, obesity, osteoporosis, mild autonomous cortisol secretion (MACS), hypercortisolemia, hypercortisolism, a sleep disorder, a mental disorder (e.g., depression, Post-Traumatic Stress Disorder (PTSD), bipolar disorder, mental fatigue, etc.), Addison's disease, long COVID, glucocorticoid withdrawal syndrome, parathyroid dysfunction, autoimmune conditions, and/or any other condition.

Examples of a characterization of an analyte level include: a single analyte level (e.g., cortisol level at a specific time, mean cortisol level, etc.), characterization of an analyte level change over time (e.g., cortisol trend, characterization of the cortisol awakening response (CAR), etc.), characterization of an analyte rhythm (e.g., diurnal cortisol rhythm), and/or any other analyte level report and/or analysis. In a specific example, the health metric can be an assessment of a probability of recurrence after pituitary surgery. The health metric can optionally be used to monitor: an analyte level, an analyze level rhythm (e.g., diurnal cortisol rhythm), a condition, a user state, and/or any other parameter. The health metric can optionally be used to aid in the diagnosis, monitoring, and/or treatment of a condition (e.g., a condition associated with cortisol, a condition associated with another analyte, etc.).

In an example, the health metric can be a score comparable to (e.g., replacing) a result of a test (e.g., a standard-of-care test). Specific examples of tests include: blood test, a urine test, a saliva test, a blood-based overnight img dexamethasone suppression test (DST), a 24-hour urine free cortisol level (UFC), late-night salivary cortisol testing (LNSC), blood (serum) cortisol test, salivary cortisol test, hair cortisol analysis, dried blood spot (DBS) cortisol test, nighttime saliva tests (NST), ACTH stimulation test, and/or any other test.

In variants, to translate cortisol levels into clinically meaningful values, empirical correlations with standard-of-care assays can optionally be established. In a specific example, clinical reference standards such as 24-hour urine free cortisol, salivary free cortisol, and total serum cortisol can be used and have well-characterized physiological relationships. Urine free cortisol reflects the unbound fraction of cortisol excreted over a 24-hour period and correlates with mean serum-free cortisol levels, particularly in cases of cortisol excess. However, because it represents an integrated rather than instantaneous measure, temporal correction factors may be required when correlating with continuous ISF-based readings. Salivary cortisol provides a non-invasive measure of the free, biologically active fraction of cortisol that diffuses from plasma into saliva. While salivary and dermal ISF cortisol levels are generally correlated, differences in assay methods and local enzymatic activity in saliva, including enzymes that interconvert cortisol and cortisone, may introduce variability that may be corrected.

In an example, the health metric can include or be informed by user-reported data (e.g., patient-reported symptoms). In an example, a user can report symptoms associated with glucocorticoid withdrawal and/or adrenal insufficiency, and the health metric can include a characterization of analyte levels indicating whether the user is likely suffering from glucocorticoid withdrawal and/or adrenal insufficiency. In an illustrative example, the health metric can show that the user does not have adrenal insufficiency, but is suffering from glucocorticoid withdrawal syndrome.

The health metric can optionally include and/or be used to determine a recommended action for a user. Examples of recommended actions include: sleep recommendation, eating recommendation, exercise recommendation, meditation recommendation, breathing recommendation, medication recommendation (e.g., recommendation to take a medication, recommendation to adjust administration of a medication, etc.), diagnostic testing recommendation, therapy recommendation, and/or any other health action. Recommended actions can be a one-time recommendation, a daily activity recommendation, and/or a recommendation for any other timescale. The recommended action can optionally be made to a user to achieve a target (e.g., a target cortisol level, a target cortisol rhythm, etc.). In a specific example, the target can be a typical healthy cortisol diurnal rhythm. The recommended action can optionally be determined in response to a trigger. Examples of triggers include: an analyte level (e.g., cortisol level) is above or below a threshold value (e.g., the analyte level is above a maximum acceptable level or the analyte level is below a minimum acceptable level), an analyte level is increasing or decreasing at a rate that is above or below a threshold value, a biomarker (e.g., heart rate, HRV, etc.) is above or below a threshold value, and/or any other trigger.

The health metric can optionally include and/or be used to determine an adjustment to the administration of a drug. For example, the method can include adjusting an administration of a drug to the user based on the health metric. The drug is preferably an analyte-modulating drug (e.g., a cortisol-modulating drug), but can alternatively be any other drug. Examples of adjustments to the administration of a drug can include adjusting: drug titration, a timing of dosing, a dosage, a number of doses per day, a tapering protocol, and/or any other drug dosing parameters. In an example, the adjustment can be based on a target (e.g., the adjustment can be determined to attain a target). Specific examples of a target include: a target analyte level (e.g., cortisol level), a target analyte rhythm (e.g., cortisol rhythm), a target analyte rate of change, a target ratio between two analytes, and/or any other target. In a specific example, the adjustment to the administration of the drug can be determined in a closed-loop feedback system (e.g., iteratively determining analyte levels and the adjustment to an analyte-modulating drug based on the analyte levels). In a specific example, the method can include a closed loop for therapeutic titration for an analyte-modulating drug (e.g., a cortisol-modulating drug). In an illustrative example, a first time series of cortisol levels can be determined, an adjustment to the administration of a cortisol-modulating drug can be determined based on the first time series of cortisol levels and a target, an updated time series of cortisol levels can be determined, and an updated adjustment to the administration of a cortisol-modulating drug can be determined based on the updated time series of cortisol levels and the target.

The health metric can be determined (e.g., characterized): using a model, using a set of heuristics, manually, and/or otherwise determined. Inputs to the model can include: one or more analyte levels (e.g., one or more sets of analyte levels, determined via S100), one or more features (e.g., determined via S200), data (e.g., biomarker data, environmental data, patient-reported data, any data received via S400, etc.), and/or any other suitable inputs. Outputs from the model can include the health metric and/or any other suitable outputs. The model (e.g., a health metric model) can use classical or traditional approaches, machine learning approaches, and/or other approaches. The model can include regression (e.g., linear regression, non-linear regression, logistic regression, etc.), decision tree, LSA, clustering, association rules, dimensionality reduction (e.g., PCA, t-SNE, LDA, etc.), neural networks (e.g., CNN, DNN, CAN, LSTM, RNN, encoders, decoders, deep learning models, transformers, etc.), ensemble methods, optimization methods, classification, rules, heuristics, equations (e.g., weighted equations, etc.), selection (e.g., from a library), regularization methods (e.g., ridge regression), Bayesian methods (e.g., Naiive Bayes, Markov), instance-based methods (e.g., nearest neighbor), kernel methods, support vectors (e.g., SVM, SVC, etc.), statistical methods (e.g., probability), comparison methods (e.g., matching, distance metrics, thresholds, etc.), deterministics, genetic programs, and/or any other suitable architecture. The model can include (e.g., be constructed using) a set of input layers, output layers, and hidden layers (e.g., connected in series, such as in a feed forward network; connected with a feedback loop between the output and the input, such as in a recurrent neural network; etc.; wherein the layer weights and/or connections can be learned through training); a set of connected convolution layers (e.g., in a CNN); a set of attention layers (e.g., cross-attention layers, self-attention layers, etc.); and/or have any other suitable architecture. The model can include less than 10, tens, hundreds, thousands, tens of thousands, hundreds of thousands, and/or any other number of parameters (e.g., weights, biases, etc.). The model can optionally extract data features (e.g., feature values, feature vectors, high-dimensional features, embeddings in a high-dimensional space with hundreds or thousands of dimensions, human-unintelligible features, etc.) from the input data, and determine the output based on the extracted features. However, the model can otherwise determine the health metric based on the input data.

The model can be trained, learned, fit, predetermined, and/or can be otherwise determined. The model can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated. The model can be learned or trained on: labeled data (e.g., data labeled with the target label), unlabeled data, positive training sets (e.g., a set of data with true positive labels, negative training sets (e.g., a set of data with true negative labels), and/or any other suitable set of data. The model can optionally be validated, verified, reinforced, calibrated, or otherwise updated based on newly received, up-to-date measurements; past measurements recorded during the operating session; historic measurements recorded during past operating sessions; or be updated based on any other suitable data.

However, the health metric can be otherwise determined.

The method can optionally include receiving data S400, which functions to receive biomarker data, user inputs (e.g., patient-reported data), temperature data, analyte level calibration data, and/or any other data for use in all or parts of the method. The data can be used for: extracting the set of features, determining the health metric, patient monitoring, performing a correction and/or calibration, processing data (e.g., processing signals received from the reader, processing analyte levels, etc.), and/or otherwise used.

In a first variant, the data can include measured data. The measured data can include biomarker data, environmental data, system data, and/or any other data. Examples of measured data can include: heart rate, heart rate variability (HRV), blood pressure, respiration rate, sleep tracking data, photoplethysmography data, motion data, glucose data, light, derived metrics thereof (e.g., blood oxygenation (SpO₂) derived from photoplethysmography data), and/or any other measurements. The measured data can be received from one or more sensors. In a specific example, the sensor can be a wearable sensor. The sensor(s) can be a sensor in the system, a sensor in a separate device (e.g., user device, phone, watch, ring, bracelet, headband, cranial device, finger probe, glasses, bed, mattress, mattress cover, camera, radar, Wi-Fi, motion, etc.), brain-computer interface device, and/or any other sensor. Examples of sensors include: accelerometers, gyroscopes, motion trackers, Wi-Fi, radar, location tracker, scales, oxygen monitors, heart rate monitors, heart rhythm monitors (e.g., electrocardiogram, ambulatory cardiac monitoring, etc.), blood pressure monitors (e.g., ambulatory, non-ambulatory, invasive), respiratory rate monitors, oxygen and/or pulse oximetry sensors, electroencephalogram (EEG) monitors, cerebral tissue oximetry sensors, functional near-infrared spectroscopy, end-tidal CO₂ sensors, light sensors (e.g., to measure light at various times of the day), glucose monitors (e.g., continuous glucose monitors), and/or any other sensors.

In a second variant, the data can include one or more user inputs. In a specific example, the user input(s) can include user-reported data (e.g., patient-reported data). Examples of user-reported data include: symptoms, meals, sleep, stress, travel, exercise, medication, and/or any other data input by a user.

In a third variant, the data can include temperature data. In a first example, the system can include a temperature sensor (e.g., on an optics board, in a flow cell, etc.), wherein temperature measurements can be used for system calibration and/or analyte binding probe calibration (e.g., correcting received signals based on the measured temperature). In variants, applying temperature corrections can account for differences in sensor signal (e.g., changes in the response from an aptamer switch and/or potential changes in binding affinity).

In a fourth variant, the data can include analyte level calibration data. For example, a calibration factor (e.g., a single value, a curve, etc.) can be used to translate signal(s) received from a set of analyte binding probes to an analyte level. The calibration factor can be determined and/or adjusted using the analyte level calibration data. In an example, the analyte level calibration data includes an analyte level determined via a serum test (e.g., blood test, saliva test, etc.). In an illustrative example, a user can undergo a serum test concurrent with acquiring signals from the set of analyte binding probes, wherein the results of the serum test can be used to calibrate the signals.

However, data can be otherwise received.

The method can optionally include transmitting data S450, which functions to provide information to the user, a physician (e.g., a designated health care provider), and/or another party (e.g., parent, authorized third-party, etc.). In a specific example, the data can be transmitted for retrospective analysis by a physician to aid in the diagnosis of a condition (e.g., adrenal disorder, etc.). Additionally or alternatively, transmitting data can function to send data to a processing system (e.g., a remote processing system). The data can include: signals (e.g., acquired by the recorder), analyte levels (e.g., a time series of analyte levels), features, health metric, biomarker data, processed and/or unprocessed data, calibrated data (e.g., after S500), and/or any other information. The data can optionally be transmitted as a report. The data can optionally be transmitted in near real-time (e.g., a physician can view a patient's data in real time).

Transmitting data can optionally include providing a notification (e.g., alert). The notification can optionally be a cloud-based notification. Examples of triggers for the notification can include: an analyte level (e.g., cortisol level) is above or below a threshold value (e.g., the analyte level is above a maximum acceptable level or the analyte level is below a minimum acceptable level), an analyte level is increasing or decreasing at a rate that is above or below a threshold value, a biomarker (e.g., heart rate, HRV, etc.) is above or below a threshold value, and/or any other trigger. The notification can include the data, the trigger (e.g., a description of the trigger for the notification), and/or any other trigger. In a specific example, the trigger can be a cortisol level below a threshold.

However, data can be otherwise transmitted.

The method can optionally include calibrating data S500, which can function to calibrate the data and/or features derived therefrom. Calibrating data can be performed after S100, after S200 (e.g., calibrating features), after S400 (e.g., after receiving temperature data), after S300, and/or at any other time. The data undergoing calibration (e.g., correction) can be: processed and/or unprocessed signals (e.g., output by the reader), features, health metrics, and/or any other data.

In a first embodiment, calibrating data can include: converting one or more switch output signals (e.g., optical, electrical, electrochemical, etc.) into analyte concentration values using one or more calibration relationships. In a second embodiment, calibrating data can include: mapping signals (e.g., reader outputs) to analyte concentration using one or more of: a calibration factor, calibration curve, calibration surface, lookup table, regression model, and/or fitted model parameters. In a third embodiment, calibrating data can include: performing calibration using probe-specific, device-specific, patient-specific, environment-specific, and/or time-varying calibration parameters. In a fourth embodiment, calibrating data can include: performing calibration prior to use, during use (e.g., in situ), periodically during wear, and/or continuously through adaptive updating. In a fifth embodiment, calibrating data can include: applying calibration as part of a signal processing pipeline configured to produce a time-series of corrected analyte concentration values. In a sixth embodiment, calibrating data can include: implementing calibration and correction using physics-based models including binding equilibrium models, kinetic rate models, and/or sensor response models. In a seventh embodiment, calibrating data can include: implementing calibration and correction using non-physics-based methods including regression models, lookup tables, spline fits, and/or machine-learning models. In an eighth embodiment, calibrating data can include: implementing hybrid models that combine a physics-based structure with empirically fitted parameters. In a ninth embodiment, calibrating data can include: updating one or more calibration parameters adaptively during use based on observed sensor behavior, reference channels, temperature measurements, and/or detected physiologic events.

In a first variant, calibrating data can include performing a temperature correction. In a specific example, the temperature correction can include correcting for temperature dependence of the reaction between an analyte and an analyte binding probe (e.g., as approximated by an Arrhenius equation). For example, performing a temperature correction can include: applying a temperature correction to a signal (e.g., the signal recorded by the reader) to produce a temperature-corrected signal, wherein the analyte level is determined based on the temperature-corrected signal. In variants, optical analyte binding probes (e.g., optical aptamers) and/or electrochemical analyte binding probes (e.g., electrochemical aptamers) can be temperature-dependent constructs. In an example, the performance of electrochemical analyte binding probes can be determined by electron transfer kinetics in specific, analyte concentration-dependent distributions of structural conformations of redox-labeled oligonucleotides. In an example, because temperature changes alter the electron transfer kinetics, the baseline signal and the signaling gain compared to baseline can also change at specific interrogation parameters (e.g., square-wave voltammetry frequency, amplitude, etc.); temperature-aware calibration of the system can optionally be used to correct for these changes. Temperature-dependent changes in performance of analyte binding probes can optionally depend on the specific analyte binding probe structure. In an example, adjustment of interrogation parameters in different temperature ranges can maintain quantitative biosensor accuracy, wherein a calibration procedure can be implemented that accounts for analyte concentration, temperature, and/or interrogation parameters for each system configuration.

In a first embodiment, performing the temperature correction can include: measuring temperature (e.g., at or near the sensing element, at or near the set of analyte binding probes, at or near the reader, etc.; via S400), and applying temperature compensation. In a specific example, this temperature compensation can reduce temperature-induced variation in analyte binding probe response (e.g., aptamer switch response). In a specific example, performing the temperature correction can include: measuring a temperature; and based on the measured temperature, applying a temperature compensation (e.g., temperature correction) to a signal (e.g., the signal recorded by the reader) to produce a temperature-corrected signal (e.g., wherein the analyte level is determined based on the temperature-corrected signal).

In a second embodiment, performing the temperature correction can include: correcting for temperature-dependent changes in one or more analyte binding probe characteristics (e.g., aptamer switch characteristics, reporter molecule characteristics, etc.). Specific examples of analyte binding probe characteristics including: baseline signal, signal gain, signal dynamic range, binding affinity and/or sensitivity (e.g., an effective dissociation constant), binding kinetics (e.g., association/dissociation rates and/or effective time constant), and/or redox reporter dynamics. In a specific example, the temperature correction for analyte binding probe characteristics can be specific to the molecular sequence(s) of the set of analyte binding probes. In an example (e.g., for an electrochemical sensor system), the temperature correction for analyte binding probe characteristics can correct for temperature dependence of redox reporter dynamics. In a specific example, the physical location of the redox reporter on the analyte binding probe can impact the redox reporter dynamics (e.g., how frequently and/or effectively the reporter can contact an electrode). In a specific example, heat can alter the conformational dynamics (e.g., stiffness and motion) of the DNA of the analyte binding probe, changing how frequently and/or effectively the reporter can contact the electrode.

In a third embodiment, performing the temperature correction can include: correcting for temperature-dependent changes in reader components and/or signal transduction components (e.g., components of the optical path in the system). In a specific example, the temperature correction can be configured to compensate for optical effects, hardware effects, and/or electronic effects, such as fluorophore brightness, quenching, lifetime, excitation source output, reader response (e.g., detector response), and/or electronic drift. In a specific example, temperature correction for hardware system components can account for channel-to-channel and/or device-to-device variances (e.g., variances due to manufacturing, variances due to the individual reader components, etc.). In a first example, the temperature correction can correct for temperature dependence of a light source in the reader (e.g., the laser changing power and/or efficiency as a function of temperature, spectral profile changing spatially and/or wavelength as a function of temperature, etc.). In a second example, the temperature correction can correct for temperature causing components in the optical path (e.g., from a light source in the reader to the set of analyte binding probes, and from the set of analyte binding probes to the detector in the reader) to change (e.g., changing efficiency of filters in the reader, materials expanding or contracting such that the optical path changes, etc.). In a third example, the temperature correction can correct for temperature dependence of the detector in the reader. In a fourth example (e.g., for an electrochemical sensor system), the temperature correction can correct for temperature dependence of electron transfer kinetics. In a specific example, temperature can modulate the electron transfer kinetics between the redox reporter and the electrode surface. In another specific example, the reorganizational energy of the redox reporter near the electrode surface can also be a temperature-dependent factor. In a fifth example (e.g., for an electrochemical sensor system), the temperature correction can correct for temperature dependence of square wave frequency. In a specific example, the effect of temperature can be coupled with the Square Wave (SW) frequency used for interrogation.

In a fourth embodiment, performing the temperature correction can include: applying a temperature-indexed calibration. In a first specific example, applying a temperature-indexed calibration can include selecting a calibration model based on measured temperature. In a second specific example, applying a temperature-indexed calibration can include interpolating between calibration models corresponding to discrete temperature values.

In a fifth embodiment, performing the temperature correction can include: implementing temperature correction using physics-based models and/or empirically fit models (e.g., lookup tables, regression, piecewise models). In a specific example, the physics-based models and/or empirically fit models can include model(s) that treat temperature as an explicit input variable.

In a sixth embodiment, performing the temperature correction can include a combination of two or more of the previous embodiments. For example, the temperature correction can include a temperature correction for the reader and a temperature correction for the set of aptamer binding probes.

Performing the temperature correction can optionally include determining a set of parameters characterizing the system (e.g., characterizing one or more of: the reader, the optical path, the set of analyte binding probes etc.), and determining the temperature correction based on the set of parameters. In a first example, all or a portion of the system (e.g., the reader) can be placed in an environmental chamber, wherein temperature and/or humidity can be controlled to determine the temperature correction. In a specific example, a known analyte level can be measured at different environmental temperatures to determine the temperature correction (e.g., for hardware components and/or the set of analyte binding probes). A flow-valve setup can optionally be used to test the response of the set of analyte binding probes to various analyte concentrations at the various temperatures. In a specific example, the final signal gains can be analyzed to determine the equilibration and/or thermodynamics at various temperatures. In another specific example, the kinetics and/or rates of change can be measured to determine the temperature correction. In a second example, individual system components can be characterized in isolation (e.g., checking the laser power of the reader, etc.). In a third example, a mock sample and/or a reflective material can be used to test the temperature response of all or a portion of the system. In a fourth example, the optical path (e.g., excitation path and/or emission path) can be tested in isolation (e.g., measuring the power output to sample at various temperatures to test the excitation pathway in isolation; using lasers with known power outputs to test the emission path in isolation; etc.).

In a second variant, calibrating data can include performing a kinetic and/or dynamic correction. In a first example, performing the kinetic and/or dynamic correction can include: correcting reader output (e.g., signals) to estimate underlying analyte concentration under non-equilibrium conditions where the analyte binding probe response (e.g., aptamer switch response) exhibits time dependence and/or lag. In a second example, performing the kinetic and/or dynamic correction can include: compensating for binding kinetics of the analyte binding probe (e.g., aptamer switch), such that the reported concentration corresponds to the underlying analyte concentration (e.g., rather than a delayed and/or attenuated sensor response). In a third example, performing the kinetic and/or dynamic correction can include: computing a corrected analyte concentration based on a time-series of measurements (e.g., rather than relying solely on a single time-point output). In a fourth example, performing the kinetic and/or dynamic correction can include: applying kinetic correction using one or more of: first-order response models, multi-state kinetic models, inverse filtering or deconvolution, state estimation (e.g., Kalman and/or Bayesian filtering), and/or learned or empirically derived dynamic models. In a fifth example, performing the kinetic and/or dynamic correction can include: estimating kinetic parameters (e.g., effective time constant, association/dissociation rates, lag parameters, etc.) during calibration and/or updating kinetic parameters during use to compensate for changing sensor response. In a sixth example, performing the kinetic and/or dynamic correction can include: applying temperature-adjusted kinetic parameters to improve accuracy of corrected concentrations during dynamic changes in analyte levels.

In a third variant, calibrating data can include performing a feature-preserving calibration. In a first example, performing the feature-preserving correction can include: performing calibration and/or correction to preserve physiologic features of an analyte time-series (e.g., including timing, slope, amplitude, and/or shape of events). In a second example, performing the feature-preserving correction can include: correcting sensor output to improve accuracy of extracted temporal features. Specific examples of temporal features can include one or more of: cortisol awakening response (CAR) amplitude, slope, and/or timing; diurnal phase, peak timing, trough timing, rhythm amplitude; pulse and/or transient detection (frequency, magnitude, duration, etc.), and/or response to events (e.g., stressors, sleep transitions, dosing, etc.). In a third example, performing the feature-preserving correction can include: applying a dynamic correction configured to reduce feature distortion caused by response lag, temperature sensitivity, mass transport (such as diffusion across a membrane to the sensing element), baseline drift, and/or sensor gain drift. In a fourth example, performing the feature-preserving correction can include: selecting a correction approach based on detected signal regimes (e.g., stable vs rapidly changing concentration). In a specific example, this can improve the fidelity of derived features.

In a fourth variant, calibrating data can include performing a reference-based and/or ratiometric correction. For example, performing the reference-based and/or ratiometric correction can include: performing a calibration using one or more reference signals. Specific examples of reference signals can include signals from one or more of: a reference fluorophore and/or reference optical channel, reference redox reporter or reference electrode, a reference sensor region lacking an analyte-binding switch, a reference analyte-binding probe, and/or an independently measured analyte expected to exhibit a different temporal profile or stability relative to the target analyte. In a first example, performing the reference-based and/or ratiometric correction can include: using reference signal(s) to normalize analyte binding probe output (e.g., aptamer switch output) and/or reduce sensitivity to drift, temperature changes, optical coupling changes, and/or hardware variability. In a second example, performing the reference-based and/or ratiometric correction can include: computing a ratiometric measurement using target and reference channels to generate a corrected signal (e.g., a corrected signal that is less sensitive to confounding factors). In a third example, performing the reference-based and/or ratiometric correction can include: using a known magnitude reference and/or internal standard to normalize scaling, correct excitation intensity variation, and/or correct detector response variation. In a fourth example, performing the reference-based and/or ratiometric correction can include: using a reference-based correction to stabilize long-term measurements and/or improve reliability of extracted physiologic features.

However, data can be otherwise calibrated.

### Cortisol Physiology and the Hypothalamic-Pituitary-Adrenal (HPA) Axis

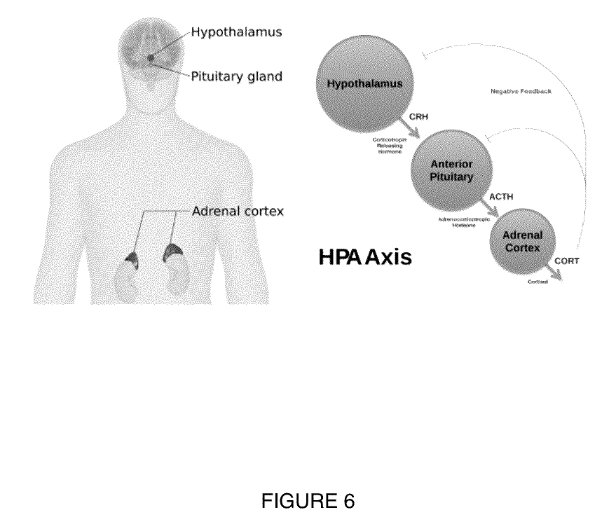

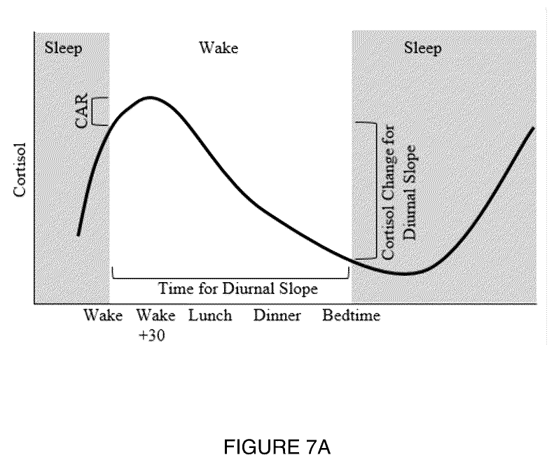

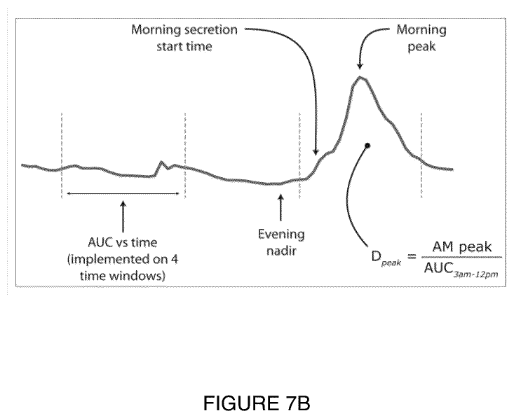

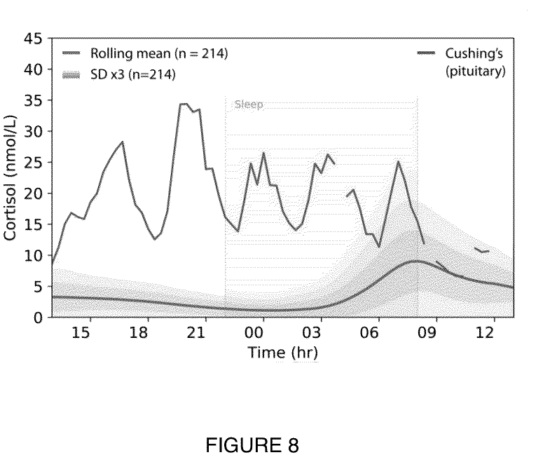

The HPA axis is a network of three organs and hormones which responds to internal and external stimuli, commonly referred to as stressors, by activating cortisol secretion (FIG. 6). The brain's hypothalamus secretes corticotropin releasing hormone (CRH) into the bloodstream, which then signals the brain's pituitary gland to secrete adrenocorticotropic hormone (ACTH). The secreted ACTH travels through the bloodstream to the adrenal cortex on top of the kidneys to activate the biosynthesis of cortisol, which enters circulation. Circulating cortisol has an inhibitory effect on CRH and ACTH production, and thus as circulating cortisol levels increase, there is negative feedback applied to the HPA axis that decreases CRH and ACTH, eventually leading to suppression of cortisol biosynthesis. This regulatory negative feedback loop leads to a characteristic cyclical and pulsatile behavior in cortisol levels, observed as multiple cortisol spikes throughout the day. A wide range of HPA axis disorders that affect CRH, ACTH, or cortisol levels lead to disruption of this natural pulsatile and cyclical behavior. Furthermore, cortisol levels follow a characteristic diurnal rhythm, with a morning cortisol peak that is driven by the brain's central clock, followed by the aforementioned pulses with diminishing spike amplitude throughout the day, leading to a nighttime cortisol trough (FIG. 7A and FIG. 7B). A wide range of HPA axis disorders lead to deviations from this healthy circadian rhythm (FIG. 8).

Outside of the HPA axis, cortisol has a broad range of downstream effects on physiology that are mediated by its interaction with glucocorticoid receptors (GR) that are present throughout almost all tissues and cells within the body. Once activated by corticosteroid ligands, a number of GR variants induce or repress the transcription of thousands of genes through direct binding to DNA response elements, physically associating with other transcription factors, or both. Cortisol-bound GR in the cytosol or membrane can also trigger more rapid, non-genomic effects by activating other proteins. Thus, cortisol levels have a broad range of metabolic effects. Cortisol increases blood glucose delivery to the brain. Cortisol reduces glycogen synthesis and increases gluconeogenesis (synthesis of glucose from lactate) in the liver. In muscle cells, cortisol decreases glucose uptake and consumption and increases protein degradation to supply gluconeogenesis with glucogenic amino acids. In adipose tissues, cortisol increases lipolysis (a catabolic process that results in the release of glycerol and free fatty acids). Cortisol also acts on the pancreas to suppress both insulin and glucagon secretion. Cortisol suppresses insulin secretion from the beta cells in the pancreas. Cortisol suppresses glucagon secretion from the pancreas. Thus, dysregulated cortisol levels have substantial interfering effects on healthy metabolic processes within the body. Beyond metabolism, cortisol levels regulate the immune system, inflammation, mental health and stress, and sleep, and cortisol levels outside of the normal physiological range have negative impacts on all of these key health factors.

### Diagnosis and Treatment of Cushing Syndrome and Cushing Disease

Cushing disease (CD) is a state of hypercortisolemia (elevated cortisol levels) that is caused by a pituitary tumor that overproduces ACTH, leading to elevated cortisol biosynthesis. Signs and symptoms of CD are weight gain (especially in the face and abdomen), fatty deposits between the shoulder blades, infertility, diabetes, hypertension, hirsutism in women, proximal muscle weakness, and osteoporosis. Cushing disease has an incidence of ~40 per million in the US. Cushing syndrome (CS) is characterized by general hypercortisolemia, regardless of cause. Symptoms can be similar to CD. As defined here, CD is a subset of Cushing syndrome (CS), which includes other sources of hypercortisolemia beyond pituitary tumors, including ectopic sources of ACTH production (those not located in the pituitary gland). CD is confirmed through more etiology-focused testing (e.g. radiological imaging) after the initial CS diagnosis. The treatment for CS is usually surgery to remove the lesion (adrenal or pituitary). If the treatment does not resolve the disease, this treatment can be followed by repeat surgery, radiation therapy, or drugs (e.g. adrenal steroidogenesis inhibitors, Glucocorticoid-receptor antagonists) that either suppress cortisol production or suppress the glucocorticoid receptor response to high cortisol levels. Pharmaceutical treatment is used for ectopic sources of CS that cannot be removed surgically.

Cortisol measurements can play an important role in the diagnosis and management of CS/CD, but their utility is limited due to the constraints of current cortisol measurement technologies which are limited to single-time-point measurements. Cortisol measurements are the first step in CS/CD diagnosis, where a combination of 2-3 cortisol testing methods is used to establish hypercortisolemia. Examples of cortisol testing methods include: a blood-based overnight img dexamethasone suppression test (DST), a 24-hour urine free cortisol level (UFC), and late-night salivary cortisol testing (LNSC). The DST relies on administering img dexamethasone at night (11 μm-midnight) which suppresses cortisol production by the adrenal glands, followed by morning (8-9 am) blood sampling for cortisol measurement, where elevated cortisol above a reference range indicates hypercortisolemia. This test requires patient visits to a clinical facility for drug administration and trained personnel to obtain samples. The UFC test relies on the patient collecting all urine for 24 hours, which is then used for analytical testing, however this test has a high rate of error and low patient compliance. LNSC testing is a self-administered test where the patient collects a saliva sample at night, but not all facilities are equipped for this saliva-based test and it relies on careful timing of the test to avoid deviations due to inter-individual differences in cortisol diurnal rhythm. A CS/CD diagnosis requires at least 2 of these above tests to be performed as all tests have failure modes or lack specificity such that a single test alone cannot provide a conclusive diagnosis. Thus, the process for CS/CD testing is laborious, complex, and requires a high degree of physician expertise and involvement. This is further complicated by the existence of more complicated conditions such as cyclic CS, in which hypercortisolemia may present only periodically across months to years. Diagnostic challenges also exist in differentiating CS/CD from physiologic/non-neoplastic hypercortisolism (formerly pseudo-Cushing syndrome), which is CS-like symptoms that are not caused by a tumor, but rather by the patient's physiology or disease states that interact with the HPA axis such as obesity, polycystic ovary syndrome (PCOS), poorly controlled diabetes mellitus (DM), chronic alcoholism, and psychiatric disorders. These conditions are often differentiated by considering the patient's medical history, along with other diagnostic tests.

Currently, cortisol measurements play only a limited role in the treatment of CS/CD. In cases where surgery is performed to remove tumors causing CD, there is approximately a 50% recurrence rate. Post-surgery cortisol levels that return to normal levels, along with a reestablishment of the normal cortisol diurnal rhythm are the best predictors of non-recurrence, however there are no tests that allow longitudinal monitoring of cortisol levels and their normalization. In cases where cortisol biosynthesis modulating pharmacological interventions are used, treatment involves minimal cortisol testing and instead relies on symptoms for physician management of the patient dose. This dosing leads to a risk of cortisol oversuppression, which in turn can cause adrenal insufficiency or serious adrenal crisis.

A body-worn cortisol sensor capable of obtaining multiple cortisol measurements across time to extract features of the patient's cortisol rhythm would be highly valuable for measuring cortisol and for the diagnosis, management, and treatment of cortisol related conditions, including but not limited to CS/CD. Towards diagnosis, such a monitoring device would be capable of capturing all the information obtained through a combination of complicated cortisol tests (DST, UFC, LNSC), within a single test that can be self-administered by the patient. By obtaining temporal features of the cortisol rhythm, identification of a healthy or diseased cortisol rhythm would be a significant improvement over our current testing methods. For example, a substantial limitation of LNSC is that the test must be well-timed with the patient's nighttime cortisol trough. If the test is not taken when cortisol is at its lowest, an elevated value may be obtained and thus a false positive result may occur. In contrast, a cortisol monitor that collects readings across time would allow detection of the true daily cortisol minimum and/or maximum level. Furthermore, the characteristic diurnal cortisol rhythm, including substantial peak-to-trough differences and cyclical spiking throughout the day, is a sign of healthy cortisol levels, and a cortisol monitor would be able to detect deviations from this rhythm, for example a blunted response with smaller peak-to-trough difference or minimal cyclical spiking throughout the day. These temporal features may be much more indicative and specific for CS/CD diagnosis. Finally, a cortisol monitor could allow longer-term monitoring of cortisol levels and the diurnal rhythm. For example the user could wear such a device across multiple days, allowing more accurate consensus measurements that are not susceptible to day-to-day variability. This would also allow the integration of a img dexamethasone suppression test into the use of the device, so that both endogenous and suppressed cortisol levels could be measured to increase accuracy.

Towards CS/CD treatment, such a cortisol monitoring device would greatly improve the monitoring of patient responses to both surgical and pharmacological interventions. In the case of surgery, the device could be applied in the days to weeks following surgery to observe decreases in cortisol levels and the re-establishment of the diurnal rhythm. Lack of diurnal variation & elevated nighttime levels in cortisol have been identified as an indicator of recurrence, but current cortisol tests cannot capture these features as they cannot be applied while the patient is asleep. However, a cortisol monitor could capture these features. Longitudinal cortisol levels (out to 6-12 weeks post surgery) may be more predictive of recurrence than the days following surgery, and a wearable test that can be applied by the patient would make these measurements more accessible.

Since many patients travel out of state for this surgical care due to the highly specialized surgeries, there are a lot of patients who are lost to the follow up in the weeks after surgery since they cannot travel for post-operative appointments and care due to cost and inconvenience. Therefore, the follow up care often is conducted by non-specialized healthcare providers who may not be able to interpret the results of the cortisol testing. Having continuous cortisol testing that is self-administered by the patient and that can be more easily shared with the specialty team would be invaluable to ensuring a safe transition to health post-operation.

In the case of pharmaceutical management of CS/CD, a cortisol monitoring device would provide a method to directly monitor the desired suppression of patient cortisol levels when adrenal steroidogenesis inhibitors are prescribed. This would be a substantial advance, as current dosing approaches do not provide insight into the resulting cortisol levels, and require careful management to achieve cortisol suppression while avoiding oversuppression that leads to adrenal crisis. A monitor could be worn by the patient while they are prescribed a cortisol-modulating medication, and the dosage could be periodically modified to ensure they maintain a healthy cortisol level. Furthermore, the timing of doses could be adjusted, along with the dosing of any glucocorticoid replacements, to better replicate the natural cortisol diurnal rhythm. The inability to measure the cortisol profile throughout a day using conventional technologies has led to dosing approaches that focus on suppressing overall cortisol production, and ignore the inherent cortisol rhythm that needs to be restored. If cortisol production is suppressed, but the cortisol rhythm remains dysregulated, the patient can suffer side effects and have difficulty restoring normalized bodily functions. The suppression of cortisol production without restoration of a healthy rhythm is thought to be one of the causes of glucocorticoid withdrawal syndrome (GWS).

The most significant complication of any intervention (surgical or pharmacologic) is glucocorticoid withdrawal syndrome (GWS). Patients experience life-altering symptoms associated with the lowering of cortisol levels. These can be so debilitating that patients are often recurrently hospitalized and drop out of life, work, etc. This has been a clinical conundrum that is well documented but there is no clear understanding of the underlying mechanism of this due to our inability to measure cortisol dysrhythmia. A device capable of measuring cortisol continuously would be invaluable for treating and understanding GWS.

### Treatment and Diagnosis of Mild Autonomous Cortisol Secretion (MACS)

Mild autonomous cortisol secretion (MACS) is a disease characterized by elevated cortisol levels that are not accompanied by the same overt symptoms as CS/CD, but can be accompanied by an increased risk of a number of comorbidities (e.g. diabetes and cardiovascular disease), increased mortality, and decreased quality of life. MACS is caused by the presence of adrenal adenoma, an adrenal tumor that synthesizes cortisol independently of ACTH levels, leading to consistent elevation. MACS is rapidly gaining awareness as a prevalent condition, with an estimated prevalence of >2% in the adult population. Because MACS does not have overt symptoms, it is often incidentally diagnosed upon incidental findings of adrenal adenoma in CT scans, where ~5% of all CT scans performed indicate an adrenal adenoma and ~50% of these cases exhibit MACS. The same diagnostic standards used for CS/CD are also used to diagnose MACS. The current standard of care is the 1 mg dexamethasone suppression test, as described above for the diagnosis of CS/CD. Although patients with MACS fail the diagnostic tests, they exhibit less overt or severe comorbid complaints and conditions associated with CS/CD.

The clinical guidelines indicate that every patient with an adrenal adenoma needs a thorough clinical and endocrine work-up to exclude hormone excess including the measurement of plasma or urinary metanephrines and a 1-mg overnight dexamethasone suppression test (with a cutoff value of serum cortisol≤50 nmol/L [≤1.8 μg/dL]). However, a high proportion (~75%) of patients with an incidental adrenal adenoma do not undergo a diagnostic evaluation for hypercortisolemia and/or MACS. This is due, in part, to the limited availability of cortisol testing through the DST, as well as the complexity of administering and interpreting cortisol tests. Access to more convenient cortisol monitoring via a wearable device could decrease the barriers to cortisol testing and increase the number of patients that undergo a diagnostic workup and are correctly diagnosed with MACS. MACS also results in a blunted diurnal cortisol rhythm as cortisol synthesis from the adrenal adenoma is constant and not mediated by ACTH secretion which has a diurnal rhythm. In MACS the blunted rhythm often leads to abnormal DST and/or salivary cortisol test results, but can result in a normal 24-hour urine test. This can be a distinction between MACS and CD/CS because some regulatory bodies mandate an abnormal 24-hour urine for diagnosis and treatment with cortisol-synthesis blocking medications a priori. A continuous cortisol monitoring device would be invaluable for the diagnosis of MACS since it could detect changes in the cortisol rhythm, such as blunted rhythm, that cannot be directly monitored using conventional tests. MACS is treated similarly to CD/CS as mentioned above, with surgical intervention to remove the MACS-causing tumor considered as the first intervention, and pharmaceutical interventions to decrease cortisol levels or the impact of cortisol levels used in cases where surgery cannot be done or is ineffective.

A cortisol monitoring device would be highly valuable for both diagnosis and management of MACS. The high degree of underdiagnosis for MACS is contributed to in part by the complexity of the tests required for its diagnosis. A wearable monitor would greatly reduce the complexity of administering such testing while also increasing testing accuracy as discussed in the discussion of CS/CD diagnosis above. Furthermore, because MACS exhibits a blunted diurnal cortisol rhythm with lower pulsatility, cortisol monitoring devices may achieve higher diagnostic accuracy by directly detecting this lack of pulsatility as compared to single-time-point diagnostics which cannot measure the temporal dynamics of cortisol. As discussed above in relation to CS/CD, a cortisol monitoring device would greatly improve the monitoring of patient responses to both surgical and pharmacological interventions. Such a device could be applied in the days to weeks following surgery to observe decreases in cortisol levels and the re-establishment of the diurnal rhythm by providing temporal information that cannot be provided with traditional diagnostics. Notably, clinical trials are underway for cortisol-modulating drugs for the management of MACS, and if successful these drugs will rely on accurate dosing to achieve cortisol suppression while avoiding oversuppression and adrenal insufficiency. A monitor could be worn by the patient while they are prescribed a cortisol-modulating medication, and the dosage could be periodically modified to ensure they maintain a healthy cortisol level. Furthermore, the timing of doses could be adjusted, along with the dosing of any glucocorticoid replacements, to better replicate the natural cortisol diurnal rhythm.

### Diagnosis of Adrenal Insufficiency (AI)

Adrenal Insufficiency (AI; formerly Addison's Disease) is a range of disease states of hypocortisolism (low cortisol levels) that can be caused by various factors. Examples of symptoms of adrenal insufficiency can include fatigue, weight loss, nausea, hypotension, hyperkalemia, hyponatremia, and hyperpigmentation of the skin, caused by the lack of the key metabolic and physiological effects of cortisol. Primary AI is a lack of adrenal function which leads to both low cortisol and aldosterone biosynthesis despite robust circulating ACTH levels. The cause of primary AI is most commonly autoimmune and it has a prevalence of ~50-100 per Million. Other causes of primary AI include genetic causes. Secondary AI is a lack of ACTH/pituitary function which leads to low cortisol biosynthesis within the adrenals, leading to low cortisol levels but the absence of abnormal aldosterone synthesis. Secondary AI has a diagnosed incidence of 150-280 per million, but many of these cases are related to glucocorticoid drug use that causes long term HPA axis suppression, rather than physiological deficiencies. For non-drug-induced AI, the most common treatment is glucocorticoid replacement therapy, which relies upon the administration of exogenous glucocorticoid drugs (commonly hydrocortisone or prednisone) to increase the circulating steroid levels to replace the metabolic and physiological effects of cortisol.

AI is diagnosed by failure to respond to ACTH via an ACTH stimulation test. To conduct such a test, a serum-based early-morning cortisol level can be obtained, as a healthy diurnal cortisol profile will be elevated at that time point whereas a patient with AI will not have an elevated cortisol level. Morning cortisol levels of <100 nM (serum, total) are suggestive of AI. Levels of ~100-500 nM (serum, total) may require further testing if there is clinical suspicion of AI. This would be followed by an ACTH stimulation test, which relies on dosing the patient with exogenous ACTH to test if excess ACTH can stimulate the adrenals to produce cortisol. For these tests, the thresholds are ~400 nM (serum, total) for AI. It is notable that assay-to-assay variability in reference ranges can confound the diagnosis of AI.

A cortisol monitoring device may enhance the diagnosis of AI by improving the ability to detect the true maximum cortisol levels either during the waking response, a stress response, or after ACTH stimulation during testing. Because conventional serum-based tests only measure cortisol at a single point in time, they provide no information to determine if the measurement obtained corresponds to the true physiological morning peak. Thus, the measurement may be susceptible to timing errors due to inter-individual variabilities in circadian rhythm, and false positives may occur if samples are taken at times other than the morning peak. This uncertainty leads to the need for further testing when results lie within the intermediate range of 100-500 nM. Similarly, the current standard of the ACTH stimulation test relies upon cortisol measurements at either 30—and 60-minutes post ACTH administration, however inter-individual differences in temporal response to a dose of ACTH makes the analysis complicated as peak cortisol values may be achieved at different timepoints. In contrast, for both of these cases a cortisol monitoring device would provide the entire cortisol diurnal profile, allowing detection and quantification of the maximum morning cortisol peak or maximum ACTH-dependent cortisol peak, and thus much more accurate comparison to normal values. Furthermore, a diurnal cortisol profile for AI would likely lack the characteristic pulsatile cortisol behavior that is induced by HPA axis feedback as either adrenal or pituitary function is impaired in AI. Thus the lack of these characteristic pulses combined with a low morning cortisol level may allow for higher specificity detection of AI.

### Glucocorticoid Drug Dosing-Glucocorticoid Replacement Therapy for Adrenal Insufficiency

In diagnosed cases of AI, the standard treatment is glucocorticoid (GC) replacement therapy, where the patient is prescribed daily doses of either cortisol (hydrocortisone) or a GC with similar metabolic and physiological effects. Some examples of GCs used for replacement therapy include, but are not limited to, prednisone, dexamethasone, and methylprednisolone. The goal of this therapy is to provide a physiological level of GC stimulus. A variety of drugs and dosing regimens are used, and they need to be controlled and optimized to best match the physiological diurnal rhythm of the patient if possible, and also to avoid over/under-dosing. Common approaches are multiple (1-3) hydrocortisone (cortisol) doses daily, but prednisolone once daily is also used due to it having a longer half-life of effects. Patients receiving GCs often need to be dose-adjusted for sick days or major stresses (e.g. surgery) to replicate the increase in cortisol that would occur in these cases in healthy patients. Currently the adjustment of dosing for these drugs is done by physicians based on monitoring of the key symptoms of AI. This management is typically complex, and requires physicians to take into account reported daily activity patterns and energy levels of patients, and can be confounded by many factors. Due to the lack of cortisol monitoring devices that can measure cortisol periodically throughout the day, the dosing is often reliant on subjective judgment from the patients based on their day-to-day activity and behavior. As a result, there is substantial risk to the patient due to this variability of dosing behavior as both over- and under-dosing have serious complications as the patient can enter into Cushing syndrome symptoms, or fall back into AI.

The lack of ability to directly monitor this dosing based on circulating cortisol levels is due to a lack of cost-effective, broadly available methods to monitor this cortisol level across multiple timepoints. Individualized dosing cannot currently be informed using cortisol-based testing due to the labor intensive and expensive nature of lab-based cortisol tests. A cortisol monitoring device would be of substantial benefit to the accurate dosing of GC replacement drugs, as it would offer the ability to monitor the dosage of glucocorticoid replacement therapy and optimize patient regimens to maintain time in a healthy cortisol range, which would help avoid the serious effects of over- or under-dosing. Furthermore, because a cortisol monitoring device would allow collection of data throughout the day, these devices would aid in developing GC dosing regimens to reconstruct a natural diurnal rhythm that matches a healthy cortisol diurnal rhythm for the patient, including adjusting the number, timing, or size of doses throughout the day. The cortisol monitoring device could also be used to enable more precise stress dosing. Currently patients are often given generic stress dosing recommendations based-off subjective judgements from the patient. A continuous monitor with patient specific historical cortisol data would allow patients and clinicians to make better informed recommendations of therapy to avoid both over and under treatment. Such a monitor may also provide alerts or allow controlled increases of dosing in cases of illness or major stresses where higher cortisol levels are needed. In some embodiments, the device is configured to measure the exogenously taken GC (either alone or in addition to cortisol) in order to aid in effective dosing and/or management of a disease.

### Glucocorticoid Drug Dosing-Managing Tapering After Glucocorticoid Use

GC drugs are commonly prescribed for their anti-inflammatory and immunosuppressive properties, however long term GC use (and thus high GC circulating levels) induces lasting negative feedback on CRH and ACTH production, eventually leading to atrophy of adrenals. Thus, when GC drug administration is ceased, CRH and ACTH levels do not immediately recover and cortisol production is low, leading to glucocorticoid induced-AI (GI-AI). This poses a high risk to patients using long-term GCs as the resulting GI-AI suppression can lead to adrenal crisis alongside the typical symptoms of AI. In the US, the total population prevalence of long-term oral glucocorticoid use is estimated to be 1.2% and the absolute risk of GI-AI within this population is 48.7%. The total population prevalence of GI-AI from long-term oral glucocorticoids alone is estimated to be 0.6% of the total US population.

Thus, for this large number of patients, careful tapering off of GC drugs can enable normal HPA axis function to be regained without substantial deficiencies in circulating cortisol levels. This poses a challenge for monitoring the tapered dose, as keeping GC levels too high can risk CS or failure to taper, while dropping doses too quickly may cause AI. GC dosing and timing of drug also matters in terms of how it applies negative feedback to the HPA axis and how much it disrupts the natural circadian rhythm. The current approach to the tapering process requires complex physician intervention and relies upon withholding the GC drug for 24 hours so its effects can subside then performing a morning cortisol test to monitor if normal cortisol secretion from the HPA axis has returned to above a reference level. A cortisol monitoring device could greatly improve the accuracy of the tapering process by evaluating the dynamics of the resumption of cortisol secretion from the HPA axis after drug cessation. A wearable, portable device may make the repeated morning cortisol measurements required for taper monitoring much easier to perform, as well as improving the temporal accuracy in capturing the exact timing and value of the morning cortisol peak. This may allow patients to cease administration of the drug for a shorter period of time than 24 hours, within which a cortisol monitor could detect either the presence of suitable endogenous cortisol levels which indicate the drug can be further tapered, or the lack of endogenous cortisol which suggests the tapering process may need to be slowed and that the patient should not continue ceasing GC dosing. Furthermore, a cortisol monitoring device would allow collection of data throughout the day with which to optimize GC dosing regimens to reconstruct a natural diurnal rhythm that matches a healthy cortisol diurnal rhythm for the patient, including adjusting the number, timing, or size of doses throughout the day. This may reduce HPA axis negative feedback and improve the ability to resume normal cortisol production during drug tapering.

Supplementing, Evaluating, and/or Replacing Dexamethasone Suppression Testing by Measuring Dexamethasone

The Dexamethasone Suppression Test (DST), despite it being the standard-of-care dynamic test used to assess the entirety of the HPA axis, still has several challenges and limitations. In the DST, the patient takes a single dose of dexamethasone at 11 μm (img in low-dose testing, 8 mg in high-dose testing) and then a serum (total) cortisol measurement is drawn at 8 am. For some individuals, they metabolize dexamethasone differently, leading to false positives or false negatives. Additionally, cortisol metabolism differences due to genetics or liver enzyme activity can alter the expected suppression. Chronic stress, depression, and obesity can cause partial cortisol suppression, mimicking mild Cushing syndrome which has historically been labeled pseudo-Cushing. Alcohol use disorder and severe illness can also blunt the expected cortisol suppression. If Cyclical Cushing syndrome (CSS) is occurring but is not in an active hypercortisolism phase, the results may be falsely negative. Some ectopic ACTH-secreting tumors (e.g., lung carcinoid tumors) may partially suppress cortisol, leading to confusion in differentiation. Some drugs that increase dexamethasone metabolism can cause falsely elevated cortisol by reducing dexamethasone activity. High-dose oral estrogen therapy in older forms of contraceptives increases cortisol-binding globulin (CBG), artificially raising serum total cortisol levels. Patients could forget or mistime their dexamethasone dose, leading to inaccurate test results, and minor delays in sample collection can cause misleading readings. Mild or early-stage Cushing disease or syndrome may not show clear suppression patterns, leading to ambiguous results. In a first specific example, monitoring cortisol and dexamethasone can be used to verify a user has taken dexamethasone (e.g., to confirm the validity of DST). In a second specific example, monitoring cortisol and dexamethasone can be used to determine a DST score (e.g., a score comparable to the DST score the user would receive if they took the DST using previous methods). In a third specific example, monitoring cortisol and dexamethasone can be used to titrate the amount of dexamethasone the user is taking for an accurate DST. In a fourth specific example, monitoring cortisol and dexamethasone can be used to characterize how a patient is metabolizing dexamethasone (e.g., to provide a more accurate diagnostic interpretation of the DST). In a fifth specific example, monitoring cortisol and dexamethasone can be used to characterize how dexamethasone is suppressing cortisol in the user (e.g., to provide a more accurate diagnostic interpretation of the DST).

### Diagnosis and Management of Hypercortisolism in Diabetes

Diabetes is a chronic metabolic condition characterized by elevated blood glucose levels that occur when the body either does not produce enough insulin (type 1 diabetes) or cannot effectively use the insulin it produces (type 2 diabetes). Insulin is a hormone that allows glucose from the bloodstream to enter cells for energy, and when this process is impaired, glucose accumulates in the blood, leading to long-term damage to organs and tissues. Cortisol is thought to be related to diabetes because it raises blood glucose levels by stimulating glucose production in the liver and reducing insulin sensitivity in peripheral tissues. While this response is helpful in short-term stress, chronically elevated cortisol may contribute to persistent insulin resistance and increased blood sugar levels, thereby increasing the risk of developing type 2 diabetes or worsening glucose control in people who already have the disease.

In people with endogenous cortisol excess (e.g., Cushing syndrome, Cushing disease), abnormalities of glucose metabolism (impaired glucose tolerance and/or diabetes) are frequent, consistent with cortisol's strong diabetogenic effects. Hypercortisolism can drive both insulin resistance and beta-cell dysfunction, which together produce hyperglycemia.

The scientific literature suggests a significant prevalence of hypercortisolism in difficult-to-control type 2 diabetes, which suggests that biochemical cortisol excess may be relatively common in specific high-risk subgroups (e.g., treatment-refractory patients) and may contribute to poor glycemic control.

Therefore, the ability to measure cortisol in patients with suspected diabetes, pre-diabetes, and diabetes can aid in the diagnosis and management of hypercortisolism in patient with diabetes or at risk of diabetes. Identifying hypercortisolism in these patients may lead to targeted intervention with cortisol-modulating pharmaceuticals or lifestyle changes to treat existing diabetes or potentially to prevent continued evolution of early stages of the disease. The intervention and titration of these interventions can be assessed with cortisol measurement and monitoring.

### Diagnosis and Management of Hypercortisolism in Hypertension

Hypertension is a chronic condition in which blood pressure in the arteries is persistently elevated, increasing the risk of cardiovascular disease, stroke, kidney disease, and other complications. Blood pressure is regulated by a complex interaction of the heart, blood vessels, kidneys, and hormones. Cortisol is thought to contribute to hypertension through several mechanisms. Chronically elevated cortisol can increase blood pressure by enhancing the sensitivity of blood vessels to vasoconstrictors, promoting sodium and fluid retention through mineralocorticoid receptor activation, and impairing normal endothelial function. Over time, sustained cortisol excess may therefore contribute to the development or worsening of hypertension, even though cortisol is not typically the sole cause.

Hypertension is very common in overt endogenous hypercortisolism (e.g., Cushing syndrome) suggesting that Cushing syndrome is a contributor to the different causes of secondary hypertension. Patients with mild autonomous cortisol secretion (MACS) from adrenal incidentalomas have higher cardiometabolic comorbidity burden, including hypertension, compared with those with nonfunctioning adrenal incidentalomas.

The scientific literature suggests a significant prevalence of hypercortisolism in treatment resistant hypertension, which suggests that biochemical cortisol excess may be relatively common in specific high-risk subgroups and may contribute to hypertension through the mechanisms described above. Treating cortisol excess with surgical or medical therapy targeting the source or cortisol action typically lowers blood pressure.

Therefore, the ability to measure cortisol in patients with hypertension or with hypertension risk factors can aid in the diagnosis and management of hypercortisolism in patient with hypertension or at risk of hypertension. Identifying hypercortisolism in these patients may lead to targeted intervention with cortisol-modulating pharmaceuticals or lifestyle changes to treat existing hypercortisolism or potentially to prevent continued evolution of early stages of hypertension. The intervention and titration of these interventions can be assessed with cortisol measurement and monitoring.

### Diagnosis and Management of Obesity

Obesity is a complex, chronic disease characterized by excess body fat accumulation that results from long-term imbalance between energy intake and expenditure, influenced by genetics, environment, behavior, and hormonal regulation. Cortisol plays a role in obesity primarily through its effects on energy metabolism, appetite regulation, and fat distribution. When cortisol levels are chronically elevated, it increases glucose production in the liver and raises insulin levels, which together favor fat storage rather than fat breakdown. Cortisol also stimulates appetite and cravings for energy-dense, high-sugar foods, reinforcing positive energy balance. Adipose tissue (e.g., visceral fat) can express high levels of glucocorticoid receptors and the enzyme 11β-hydroxysteroid dehydrogenase type 1 (11β-HSD1), which locally converts inactive cortisone into active cortisol. This local cortisol amplification promotes adipocyte differentiation and lipid accumulation, creating a feed-forward loop that preferentially expands abdominal fat.

Therefore, the ability to measure cortisol in patients with weight gain and obesity can aid in the diagnosis and management of hypercortisolism in these patients. Identifying hypercortisolism in these patients may lead to targeted intervention with cortisol-modulating pharmaceuticals or lifestyle changes to treat weight gain and existing obesity. The intervention and titration of these interventions can be assessed with cortisol measurement and monitoring.

### Diagnosis and Management of Mental Health Conditions

Cortisol can play a role in mental health because it is central to the body's stress response and directly affects brain function. In the short term, cortisol helps individuals cope with stress by increasing alertness and mobilizing energy, but chronically elevated or dysregulated cortisol levels are thought to contribute to mental health disorders. Prolonged cortisol exposure can alter neurotransmitter systems and affect brain regions such as the hippocampus, amygdala, and prefrontal cortex, which are involved in mood regulation, memory, and emotional control. This dysregulation has been associated with conditions such as depression, anxiety, cognitive impairment, and sleep disturbances). While cortisol is not the sole cause of mental illness, long-term stress and abnormal cortisol signaling are believed to increase vulnerability to mental health problems and worsen symptom severity.

Therefore, the ability to measure cortisol in patients with mental health conditions (e.g., depression, anxiety, cognitive impairment, and sleep disturbances) can aid in the diagnosis and management of hypercortisolism in these patients. Identifying hypercortisolism in these patients may lead to targeted intervention to treat patients with mental health conditions. The intervention and titration of these interventions can be assessed with cortisol measurement and monitoring.

### Diagnosis and Management of Osteoporosis and Osteopenia

Osteopenia and osteoporosis are conditions characterized by reduced bone mineral density, with osteopenia representing an intermediate stage of bone loss and osteoporosis reflecting more severe skeletal weakening and a higher risk of fractures. Healthy bone is maintained through a balance between bone formation and bone resorption, and disruption of this balance leads to progressive loss of bone strength. Prolonged cortisol excess from endogenous or exogenous (e.g., long-term glucocorticoid therapy) sources shifts bone remodeling toward net bone loss, increasing fracture risk even when changes in bone density appear modest.

Scientific literature supports that endogenous cortisol excess and therapeutic glucocorticoids are major, well-established causes of secondary osteoporosis and fragility fractures-often with fracture risk that can be out of proportion to bone mineral density (BMD). In Cushing disease and syndrome, bone impairment is driven largely by deterioration of bone quality and vertebral fractures can be common at diagnosis and may persist even after biochemical cure. In many cases, vertebral or other fractures can be the cardinal sign event that leads to a diagnosis of Cushing syndrome. Mechanistically, both endogenous cortisol excess and exogenous glucocorticoids shift bone remodeling toward loss by strongly suppressing bone formation (direct effects on osteoblast lineage and osteocytes, including reduced differentiation and increased apoptosis, and reducing calcium absorption while increasing calcium loss through the kidneys) with additional effects that can increase resorption early on. Patients on glucocorticoid therapy are at significant risk of glucocorticoid-induced osteoporosis (GIOP) and fracture risk is substantially increased (especially vertebral). Guidelines recommend early fracture-risk assessment and preventive/treatment strategies.

Therefore, the ability to measure cortisol in patients with osteopenia and osteoporosis can aid in the diagnosis and management of hypercortisolism in these patients. Identifying hypercortisolism in these patients may lead to targeted intervention with cortisol-modulating pharmaceuticals to treat patients with osteopenia or osteoporosis. The intervention and titration of these interventions can be assessed with cortisol measurement and monitoring.

### The Role of Cortisol in Rheumatologic, Autoimmune, and Inflammatory Disorders and Opportunities for Diagnosis and Management of Cortisol-related Conditions

Cortisol can be a key hormone linking the endocrine and immune systems and plays a nuanced role in rheumatologic, autoimmune, and inflammatory diseases. It is produced by the adrenal glands as the end-product of the hypothalamic-pituitary-adrenal (HPA) axis and normally acts to restrain inflammation and prevent excessive immune activation. At the cellular level, cortisol suppresses transcription of pro-inflammatory cytokines such as TNF-α, IL-1, and IL-6; inhibits activation and proliferation of T lymphocytes and macrophages; and promotes anti-inflammatory pathways that help resolve immune responses after an insult.

In many chronic inflammatory and autoimmune diseases, however, the cortisol response appears inadequate relative to the inflammatory burden. Studies in conditions such as rheumatoid arthritis, systemic lupus erythematosus, inflammatory bowel disease, ankylosing spondylitis, fibromyalgia, and polymyalgia rheumatica describe a state of hypocortisolism, in which cortisol levels are normal or only modestly elevated despite high circulating inflammatory cytokines. This imbalance allows inflammation to persist and may contribute to disease flares, fatigue, and systemic symptoms. In addition, altered tissue sensitivity to cortisol-through changes in glucocorticoid receptor expression or function—can further blunt its anti-inflammatory effects at the site of disease.

The complexity of the HPA axis, however, means that within the same disease or phenotypic sub-types, there can be different cortisol responses and, therefore, that cortisol measurements have a prognostic role in disease management. For example, in rheumatoid arthritis, lower baseline daily cortisol and cortisol/ACTH ratio have been associated with poorer 2-year treatment response and failure to reach remission. In fibromyalgia, disease chronicity (long illness duration) is thought to be associated with lower cortisol while disease severity (worse pain, fatigue, cognition) is thought be associated with higher cortisol.

Therapeutically, synthetic glucocorticoids exploit cortisol's powerful anti-inflammatory properties and remain cornerstone treatments in many rheumatologic and autoimmune disorders. While highly effective at rapidly suppressing inflammation and preventing organ damage, long-term glucocorticoid exposure can lead to significant adverse effects, including osteoporosis, metabolic dysfunction, hypertension, infection risk, and adrenal suppression. As a result, modern treatment strategies emphasize using the lowest effective dose for the shortest duration possible, alongside steroid-sparing agents.

Chronically elevated cortisol from endogenous sources can have effects similar to long-term glucocorticoid therapy. Hypercortisolism can suppress protective immune functions and contribute to complications such as infection. Thus, cortisol acts as a double-edged regulator in inflammatory disease: essential for controlling immune overactivity, yet potentially harmful when its signaling is excessive or chronically altered.

Overall, cortisol plays a role as an immune modulator (e.g., tightly regulated immune modulator) whose dysregulation-whether insufficient, excessive, or resisted at the tissue level-plays a role in the pathophysiology and management of inflammatory and autoimmune diseases.

Therefore, the ability to measure cortisol in patients with rheumatologic, autoimmune, and inflammatory diseases can aid in the diagnosis and management of both hypocortisolism and hypercortisolism in these patients, whether from exogenous or endogenous sources. Additionally, continuous monitoring of cortisol can help to define disease sub-types or sub-phenotypes that have different HPA-axis characteristics. These measurements can help to initiate and titrate therapeutic interventions or behavior and lifestyle interventions to modulate (or supplement) cortisol to treat these diseases with greater specificity.

### Ultradian Cortisol Rhythm

Ultradian Cortisol Rhythm can optionally be extracted from the cortisol levels and used as a diagnostic feature. Cortisol ultradian rhythms can be evidence of intrinsic rhythms of the HPA axis which is responsible for the cortisol secretion stimulus. Observed cortisol values can be the result of rapid, pulsatile secretory bursts of ACTH that occur with a periodicity shorter than 24 hours. In humans, these pulses typically occur every 30 to 90 minutes. They can be distinct from the circadian rhythm, which modulates the overall daily pattern of cortisol secretion. The familiar circadian profile, characterized by high levels in the morning and low levels at night, can be generated through amplitude modulation of these underlying ultradian pulses (e.g., rather than through continuous secretion or changes in the frequency of the ACTH pulses). As a result, target tissues experience alternating periods of high glucocorticoid exposure and periods of low cortisol concentration (nadirs). This pulsatile pattern can be conserved across mammalian species and produces a highly dynamic endocrine signaling environment. The ultradian rhythm can be an intrinsic property of the HPA axis and can arise from delayed feedforward and feedback interactions between the hypothalamus and the adrenal cortex. Corticotroph-derived ACTH can stimulate cortisol release from the adrenal zona fasciculata, while cortisol feeds back to the hypothalamus to regulate pituitary output with a time delay that generates oscillations of measured hormone levels.

Alterations in cortisol ultradian dynamics have potential implications for disease diagnosis and management. In adrenal insufficiency, conventional replacement therapies that deliver cortisol continuously or in large oral boluses fail to reproduce physiological pulsatility. This mismatch could result in persistent fatigue, impaired quality of life, and increased metabolic risk. In contrast, restoring pulsatile delivery has been shown to improve sleep quality, working memory, and emotional processing. In metabolic disease, the pattern of cortisol exposure strongly influences glucocorticoid receptor signaling and gene expression in liver and adipose tissue. Loss of physiological pulsatility with appropriate nadirs is associated with altered glucocorticoid receptor signaling and adverse metabolic phenotypes, including insulin resistance, hepatic steatosis, and/or obesity.

Ultradian dysregulation is also relevant in psychiatric and stress-related disorders. In major depression, ultradian cortisol secretion may remain present, while non-pulsatile basal cortisol secretion can be increased, suggesting altered adrenal or hypothalamic sensitivity or loss of the trophic effects of ACTH. Stress responsiveness is also phase dependent. Stressors occurring during the rising phase of a cortisol pulse produce an amplified response, whereas stressors during the falling phase result in a blunted response. This phase dependence provides a mechanistic basis for interindividual variability in stress resilience. In endocrine hypersecretion disorders such as Cushing's syndrome, disruption of both pulsatile organization and circadian timing, including elevated levels during the normal nocturnal nadir, could serve as a diagnostic feature.

Characterization of ultradian rhythms (which cannot be measured today with single-timepoint measurements) could serve as a rich and underutilized source of physiological information. Accurate, continuous measurement of pulsatility could offer a path toward improved diagnosis, phenotyping, and management of endocrine, metabolic, and neuropsychiatric disease. Examples of features include pulse amplitude, pulse frequency, nadir depth and duration, peak-to-nadir ratio, and overall secretion density across the day. These parameters capture both the organization and regularity of HPA axis output and provide sensitivity to pathological states that preserve mean cortisol levels but alter temporal structure. Changes in these features can reflect impaired adrenal responsiveness, altered feedback sensitivity, or loss of physiological nadirs, each of which has been associated with metabolic, psychiatric, and endocrine disorders. Assessing the phase of ultradian pulses allows clinical interpretation of stress responses and therapeutic interventions, as responses depend strongly on whether perturbations occur during the rising or falling phase of a pulse. In addition, additional analytes that impact the ultradian rhythm of the HPA axis could be monitored and analyzed to provide additional insights. Together, these measurable features provide a framework for distinguishing healthy from dysregulated cortisol signaling and for guiding diagnosis, risk stratification, and treatment optimization.

### Other Applications of Cortisol Monitoring:

The ability to measure cortisol continuously would have many applications for consumers. For example, the data could be used to optimize and improve sleep, metabolic health, stress management, mental health, athletic performance, and even fertility. Many daily factors influence cortisol levels and can impact the natural rhythm and levels of cortisol in healthy subjects. Some examples include 1) the time and regularity that someone goes to sleep and wakes up, 2) exposure to light and sounds, 3) sleep aids and medications, 4) the timing and composition of meals, 5) caffeine and alcohol intake, 6) exercise, 7) mindfulness and meditation, 8) dietary supplements, and 9) other external and internal stimuli such as arguments or stressful life events. Unfortunately, although cortisol plays a vital role in many valuable aspects of daily life, since it cannot be measured using existing technologies in real time it is nearly impossible to ensure that an individual's day-to-day actions are optimized to ensure optimal health (e.g., sleep, mental health, etc). Therefore, the ability to measure cortisol periodically through a day and measure the cortisol profile over time would transform the ability of individuals to make changes in their day-to-day activities in order to ensure a healthy cortisol rhythm that maximizes their health. In particular, the analysis of the cortisol profile could provide feedback to the user with suggestions on changing their lifestyle or activities to ensure a healthy circadian rhythm of cortisol.

Sleep: Cortisol follows a circadian rhythm and when cortisol regulation is healthy the hormone helps one wake up in the morning (when the levels are high) and fall asleep at night (when the levels are low). Cortisol levels can affect melatonin levels. When this healthy cortisol rhythm becomes dysregulated or disturbed, it can impact the ability of one to sleep. For example, high cortisol levels have been linked to insomnia, waking up during the night, and less sleep overall. Various internal and external stimuli can change or disrupt the natural circadian rhythm of cortisol, and lead to suboptimal sleep. For example, vigorous exercise can lead to a spike in cortisol levels, and if the exercise is conducted late in the day it can affect the ability of a person to fall asleep since the cortisol levels remain elevated at bed time. Currently, there is no way to measure the cortisol rhythm to ensure that sleep is undisturbed. The use of a continuous cortisol monitor could allow a user to monitor their cortisol levels and adjust their daily routine to minimize disruptions of the cortisol rhythm. In turn, this could improve the sleep quality of the patient. For example, a subject could alter the timing or intensity of their workouts to ensure that the cortisol levels drop back down prior to bedtime. Other external factors such as meal timing, meditation, and exposure to light could also be modulated in order to achieve optimal cortisol regulation for sleep.

Summary of factors that can affect cortisol levels and rhythm: Sleep/Wake cycles-what time to go to bed, what time to wake up. Sleep aids-when to take them, what type of sleep aid to be taken (OTC and Rx). Meals of the day-timing windows (when to eat first and last meals, how much to eat), meal compositions, timing and dosage of caffeine and alcohol intake. Exercise-when and what type of exercise, duration of exercise, timing of intercourse. Meditation-what type, timing, and duration. Supplements-type, timing, and dosage.

Metabolic health: Metabolism is affected by the circadian clock and cortisol can be a regulator of the circadian rhythm. Cortisol levels affect hunger (ghrelin/leptin), glucose levels, and insulin resistance. Dysregulated cortisol can lead to metabolic stress and impaired metabolic action. Therefore, controlling cortisol levels and maintaining a healthy cortisol rhythm could help improve the metabolic health of wellbeing of an individual. Furthermore, dysregulated cortisol levels can affect weight loss, so restoring healthy cortisol function could aid in weight loss. A continuous cortisol monitor could be used to help an individual make lifestyle changes to promote healthy cortisol rhythm, and thus improve metabolic health.

Stress management and mental health: Numerous mental disorders or psychiatric conditions are characterized by dysregulated cortisol levels. Examples include, but are not limited, to burnout, workplace fatigue, PTSD, bipolar disorder, and depression. Evidence suggests that in many cases, restoring a healthy cortisol rhythm could help treat these disorders. Unfortunately, since conventional testing cannot measure the rhythm of cortisol, there is little action that can be taken by an individual or healthcare provider to try to maintain a natural cortisol profile. The ability to measure the concentration of cortisol over time could allow for the monitoring and treatment of these types of mental disorders. For example, a spike in cortisol could alert an individual to an upcoming panic attack or manic episode and allow the individual to take steps to mitigate the upcoming episode such as deep breathing or meditation. A cortisol monitor could also be used to track the effectiveness of various medications or interventions on restoring a healthy cortisol rhythm. The collection and analysis of cortisol levels in real-time could revolutionize our understanding, treatment, and monitoring of various mental disorders.

Exercise and recovery: Dysregulated cortisol can negatively impact an athlete's performance, training, and recovery. The ability to measure cortisol to promote a healthy cortisol rhythm could help athletes optimize their performance and reduce the effects of overtraining. It has been shown that if an athlete overtrains, cortisol levels will remain elevated the next day and the athlete can then suffer decreased performance. The use of a cortisol monitor could help athletes optimize their performance, and also modulate their training intensity to ensure that they are not over or under training (and recovering sufficiently between workouts). Cortisol could be used in a similar fashion to how heart-rate-variability (HRV) is currently being used, but would provide a more rich and physiologically specific measure of athletic recovery. Furthermore, the monitor could also be used to learn and optimize how an athlete's body responds to both mental and physical stresses.

Fertility and pregnancy: Elevated cortisol levels and dysregulation can be a cause of infertility in women by affecting normal ovulation. Evidence suggests that normalizing cortisol levels can help restore fertility in some women. Even in men, there is evidence that testosterone levels and sperm quality can go down due to increased cortisol levels. The ability to measure cortisol in real-time could help both women and men maintain healthy cortisol levels and rhythm, which in turn could improve fertility. Such a device could be used to make recommendations in conjunction with the menstrual cycle to optimize for fertility. Other biomarkers such as body temperature, progesterone, estradiol, follicle stimulating hormone (FSH), and luteinizing hormone (LH) could be used in conjunction with cortisol for optimizing fertility.

### Additional Adrenal Analytes

In addition to cortisol there are many other molecules that would be valuable to measure either by themselves or in conjunction with other analytes such as cortisol. In an example, these include molecules such as potassium and/or sodium. In another example, these include molecules such as CRH and ACTH. In such cases, additional information regarding the HPA axis and/or cortisol metabolism can be obtained from measuring these other analytes. One example is cortisone which is a steroid hormone. Cortisol is converted by the action of the enzyme corticosteroid 11-beta-dehydrogenase isozyme 2 into the inactive metabolite cortisone. By measuring cortisone, the nature of dysregulated cortisol levels can often be elucidated. For example, cortisone levels can be used to determine if patients have elevated cortisol levels due to conversion issues. Likewise, other cortisol metabolites (e.g., alpha-THF, beta-THF) and cortisone metabolites (e.g., beta-THE) can also be measured to gain insight into underlying cortisol metabolism and function. DHEA (dehydroepiandrosterone) and DHEA-S (dehydroepiandrosterone sulfate) which both play a role in the stress response, and can also be used to gain insight into the stress response. Measurement of the ratio of cortisol and DHEA may therefore provide a more accurate assessment of HPA-axis function than either hormone alone. Additionally, it can be potentially valuable to measure cortisone since it is not affected by the administration of oral hydrocortisone.

### Clinical Vignettes

Illustrative examples of the system and/or method, including specific use cases for a continuous cortisol monitor (CCM) are included below.

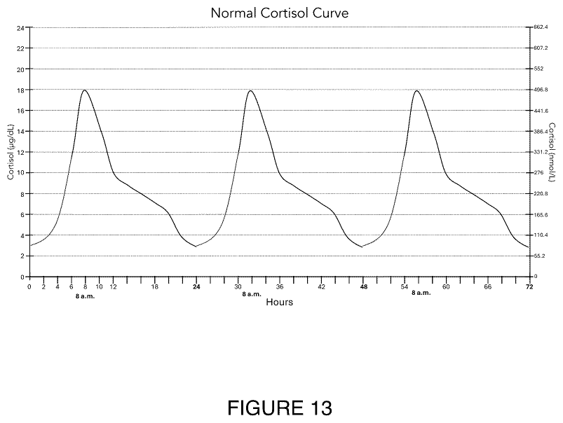

Diagnosis of a normal cortisol rhythm: A 28-year-old female has experienced fatigue and weight gain over the last several months. She has tried lifestyle changes and over-the-counter remedies without success. Her social media suggests that she should be evaluated for a cortisol abnormality, and she sees her PCP to request testing. The PCP orders 24-hour urine free cortisol, salivary testing, and dexamethasone suppression testing. These results are non-diagnostic, however the patient remains symptomatic and concerned about her cortisol. The PCP consults with an endocrinologist who recommends that the patient use a 72-hour Continuous Cortisol Monitor (CCM) for a more granular assessment of the patient's cortisol curve. The patient leaves the clinic with a monitor and self-applies the device at home. After the patient's 72-hour session, the patient's clinical data from the device (e.g., example shown in FIG. 13) are received remotely in a monitoring center and the patient returns the CCM by mail. The data are interpreted into an individualized cortisol profile by a clinical team and validated algorithms, and the results are reported to the PCP to aid in the patient's diagnosis. The report suggests that the patient's cortisol rhythm is normal. The PCP uses the CCM result, other diagnostic tests, and the patient's cumulative signs and symptoms to exclude hypercortisolism as a diagnosis. The PCP works with the patient to evaluate other potential causes of fatigue and weight gain.

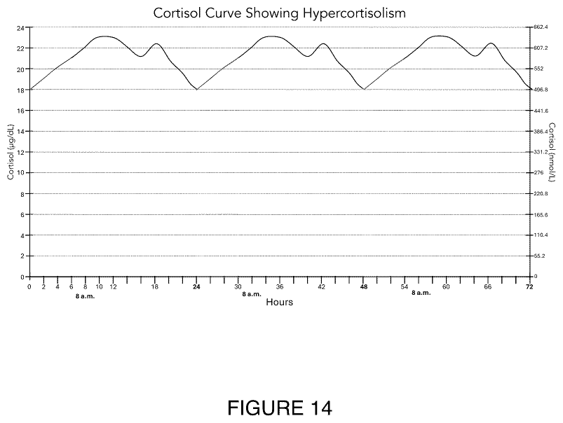

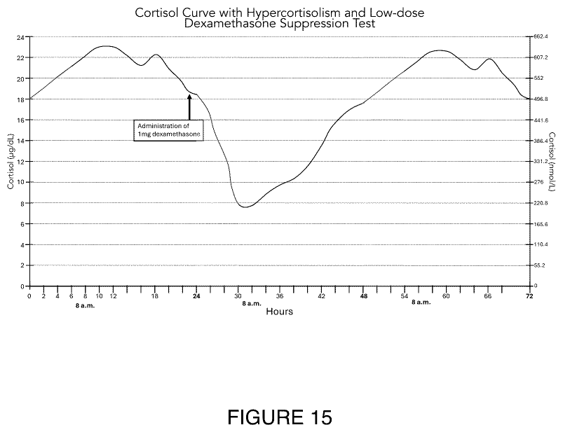

Diagnosis of Cushing syndrome: A 36-year-old female has experienced increased weight gain, fatigue, a change in her menstrual cycle, as well as up trending blood pressure. Her primary care provider is concerned about Cushing syndrome and has ordered the standard guideline recommended 24-hour urine free cortisol, salivary testing, and dexamethasone suppression testing. These results have been inconsistently abnormal and non-diagnostic, and the primary care provider (PCP) would like additional information. The PCP consults with an endocrinologist who recommends that the patient use a 72-hour Continuous Cortisol Monitor (CCM) for a more granular assessment of the patient's cortisol curve. The patient leaves the clinic with a monitor and self-applies the device at home. After the patient's 72-hour session, the patient's clinical data from the device are received remotely in a monitoring center and the patient returns the CCM by mail. The data are interpreted into an individualized cortisol profile by a clinical team and validated algorithms, and the results are reported to the PCP to aid in the patient's diagnosis. The report suggests that the patient's cortisol rhythm is abnormal (example shown in FIG. 14), and the patient is referred to the endocrinologist to discuss diagnostic findings and management options. The endocrinologist would like to run an additional low-dose dexamethasone suppression test while the patient wears the CCM (example shown in FIG. 15).

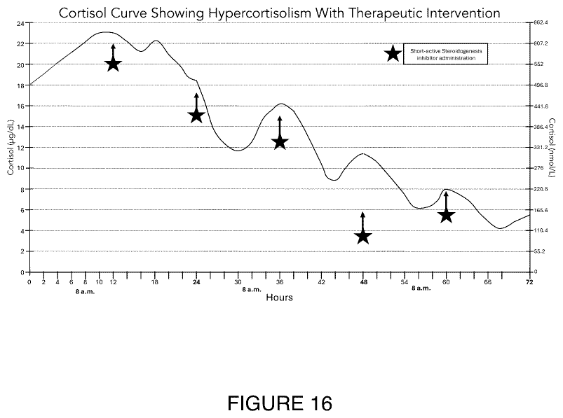

Medication (Steroidogenesis Inhibitor) Management and Titration for Cushing Syndrome: A 40-year-old female has been recently diagnosed with Cushing Syndrome by using a multi-day Continuous Cortisol Monitor (CCM) (example shown in FIG. 14). Her endocrinologist would like to start her on chronotherapy with a medication from the class of steroidogenesis inhibitors, dosed twice a day. Her clinical team would like to see how the patient responds and then titrate according to her symptoms and correlated objective clinical data. Her endocrinologist elects to apply a multi-day Continuous Cortisol Monitor (CCM) to build on the baseline individualized cortisol profile that was previously established (example shown in FIG. 14). The CCM is applied and the patient then initiates medication treatment while continuing to wear the CCM to measure the efficacy of the medication. The patient's endocrinologist opts-in to data reporting from a monitoring center (example shown in FIG. 16) and adjusts the patient's medication dosing based on the resultant curve with therapeutic intervention.

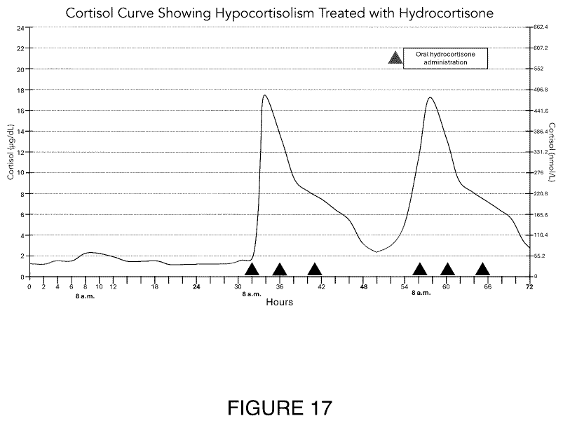

Medication Management of Permanent (Primary) Adrenal Insufficiency: A 45-year-old woman is recently diagnosed with primary adrenal insufficiency based on testing with a multi-day Continuous Cortisol Monitor (CCM) (FIG. 16). Her endocrinologist would like her to start taking hydrocortisone daily and would like a more objective measure of the efficacy of hydrocortisone beyond the patient's self-reported outcomes of episodes of fatigue and a sense of low blood pressure. The endocrinologist orders a 72-hour Continuous Cortisol Monitor (CCM) to assess her responsiveness to hydrocortisone and to adjust dosing, if needed. The patient self-applies the CCM and measures a 24-hour baseline and then begins the prescribed regimen of hydrocortisone dosed three-times per day. The endocrinologist can choose a retrospective report of the patient's 72-hour individualized cortisol curve or can log-in to near-real time data reporting from a monitoring center. The patient's individualized cortisol curve shows appropriate response to hydrocortisone treatment and restoration of a diurnal cortisol rhythm (example shown in FIG. 17).

Diabetes and Hypertension Caused by Hypercortisolism: A 60-year-old female presents with diabetes and hypertension. Her glucose has been increasingly difficult to control despite multiple medications and her A1C is up trending. In addition, she has required additional blood pressure medication. Because of this, her primary provider has referred her to an endocrinologist. Her endocrinologist is concerned that there could be a component of hypercortisolemia that is causing her conditions. The endocrinologist orders a 72-hour Continuous Cortisol Monitor (CCM) to assess her cortisol curve. The patient has a CCM applied while at the endocrinologist's office. On the night of day 2 of monitoring, the patient takes 1-mg of dexamethasone as part of a dexamethasone suppression test. The CCM provides monitoring of the patient's response to the DST. After 72 hours, the patient's clinical data from the device are received remotely in a monitoring center. The data are interpreted into an individualized cortisol profile by a clinical team and validated algorithms, and returned in a report to the endocrinologist to aid in the patient's diagnosis and subsequent management. Her cortisol curve shows excess cortisol and a disruption of the expected diurnal variation pattern, in addition to an abnormally elevated cortisol after administration of the dexamethasone as part of the DST (example shown in FIG. 15). The endocrinologist orders additional diagnostic testing to identify the location of the hypercortisolism and decides to start the patient on medical therapy for hypercortisolism.

However, the method can be otherwise performed.

### 4. System.

As shown in FIG. 2, the method can be performed using a system 10, including: a set of analyte binding probes 100, a reader 200 (e.g., an electrochemical reader, optoelectronic reader, etc.), and a processing system 300. The system can optionally include one or more of: a piercing element, a membrane, a set of flow components (e.g., a flow cell assembly, a pump, an input reservoir, an output reservoir, etc.), a set of sensors (e.g., accelerometer, motion sensor, temperature sensor, hydroscope, flow rate sensor, etc.), and/or any other suitable components. The piercing element (e.g., microdialysis probe, microneedle, needle, lumen, etc.) can optionally be configured to pierce a body surface of the user, thereby bringing the piercing element in contact with the sample of the user. The set of analyte binding probes can optionally be coupled to the reader, coupled to the piercing element, coupled to the set of flow components, and/or coupled to any other system components. The system is preferably wearable (e.g., body-worn) by a user, but can alternatively be non-wearable.

In a first variant, the system can be or include a microneedle-based system. Examples of a microneedle-based system are described in U.S. application Ser. No. 18/813,369 filed 23 Aug. 2024, U.S. application Ser. No. 18/981,409 filed 13 Dec. 2024, and/or U.S. application Ser. No. 19/098,929 filed 2 Apr. 2025, each of which is incorporated in its entirety by this reference.

In a second variant, the system can be or include a microdialysis system. Examples of a microneedle-based system are described in International PCT Application No. PCT/US2025/010333 filed 3 Jan. 2025, which is incorporated in its entirety by this reference. For example, the system can include: a pump, a reader, a piercing element (e.g., microdialysis probe, microneedle, lumen, etc.), a membrane (e.g., a semipermeable within the piercing element), an input reservoir (e.g., containing dialysate buffer), an output reservoir (e.g., to collect waste), and/or any other elements. In an example, the pump can drive fluid flow from the input reservoir, past the membrane (e.g., where the sample can equilibrate with the pumped fluid), to the output reservoir through a flow cell assembly that allows coupling between the set of analyte probes, the reader, and analytes from the sample.

In a third variant, the system can be or include a fiber optic-based system. In an example, the piercing element can include a fiber optic probe, where the tip of the fiber optic probe is located underneath the skin. The tip of the fiber optic probe can include the set of analyte binding probes (e.g., conjugated on or near the surface of the fiber optic probe), wherein the reader can transmit light to the set of analyte binding probes and/or receive light from the set of analyte binding probes via the fiber optic probe.

The processing system can function to: control one or more electronic components of the system (e.g., the reader), receive data (e.g., from the reader, from a sensor, from another device, from the user, etc.), transmit data, determining the set of analyte levels based on a set of signals, to process data, extract a set of features from the set of analyte levels, perform one or more steps of the method, and/or otherwise function. The processing system can be local (e.g., local to the reader), remote (e.g., cloud computing server, etc.), distributed, and/or otherwise arranged relative to any other system or module. The processing system can include one or more: CPUs, GPUs, TPUs, custom FPGA/ASICS, microprocessors, servers, cloud computing, and/or any other suitable components.

The reader can function to detect signals (e.g., optical signals, electrical signals, etc.) from the set of analyte binding probes. In an example, the reader can include one or more: light sources, lenses, filters, detectors, and/or any other suitable components. In a specific example, the reader (e.g., optoelectronic reader, optical measurement system, etc.) can include one or more light sources configured to direct light to the set of analyte binding probes, and one or more detectors configured to record optical signals from the set of analyte binding probes. In a specific example, the reader can record optical signals from the set of analyte binding probes (e.g., fluorescence, absorbance, changes thereof, etc.), and convert these optical signals into digital data (e.g., which can then be processed to provide real-time quantitative measurements of the analytes). Examples of readers are included in U.S. application Ser. No. 18/813,369 filed Aug. 24, 2024, U.S. application Ser. No. 18/981,409 filed 13 Dec. 2024, U.S. application Ser. No. 19/098,929 filed 2 Apr. 2025, and/or International PCT Application No. PCT/US2025/010333 filed 3 Jan. 2025, each of which is incorporated in its entirety by this reference.

### Analyte Binding Probes

The present disclosure provides sensors for analyte detection. An analyte binding probe can include polypeptide or nucleic acid sequences, antibodies, peptides, proteins, physicochemical detectors, enzymes, artificial binding proteins, and/or combinations thereof. In some embodiments, the analyte sensor (e.g., analyte binding probe) can include a polypeptide or peptide sequence such as an antibody. For example, an analyte sensor may comprise single stranded deoxyribonucleic acid, double stranded DNA (dsDNA), ribonucleic acid (RNA), nucleic acids in some cases with modified bases, and the like. The analyte sensor (e.g., analyte binding probe) may be an oligonucleotide probe and the analyte may be a complementary target nucleic acid. In another embodiment, the binding domain can be a dsDNA strand specific to a target enhancer protein target. In some embodiments, the analyte binding probe may be or include: an antigen binding probe, an aptamer, an aptamer switch, a linking moiety, an antibody-bait switch, a dual antibody switch, an antibody-aptamer switch, a reporter molecule, and/or any other molecules. In some embodiments, the analyte sensor may comprise a nucleic acid sequence comprising an aptamer.

### Antigen Binding Probes: Aptamers

Sometimes referred to as “synthetic antibodies,” aptamers may be pre-selected single-stranded oligonucleotide (e.g., DNA or RNA) or peptide molecules that bind to specific target molecules including proteins and peptides with affinities and specificities that are comparable to antibodies. These molecules can assume a variety of shapes due to their propensity to form helices and single-stranded loops with specific binding pockets, explaining their versatility in binding to diverse targets. Their specificity and characteristics are not directly determined by their primary sequence but by their tertiary structure which can be analogous to the globular shape of tRNA. Aptamers have a wide range of applications including diagnostics and therapeutics and can be chemically synthesized using known techniques. Furthermore, aptamers can offer a number of advantages over traditional antibodies including avoiding the need to specifically know the precise epitopes or biomarkers themselves. Finally, aptamers may be typically non-immunogenic, easy to synthesize, characterize, modify, and exhibit high specificity and affinity for their target antigen.

The aptamer may be nucleic acid or peptide molecules that bind to a specific target molecule. In some embodiments, binding of the target analyte to the aptamer induces conformational changes in the aptamer. In some embodiments, the aptamer may bind to various molecular targets, for example, small molecules, macromolecules, metabolites, proteins, carbohydrates, metals, nucleic acids, cells, tissues, and organisms.

By using a variety of selection techniques, aptamers can be selected to find targets, e.g., on a surface or inside a cell of interest, without the need to identify the precise biomarker or epitopes themselves. In many cases, the aptamer identification process can begin with a large random pool of oligonucleotides or peptides that are systematically subjected to negative and positive rounds of selection against a target, e.g., a protein molecule, to filter out low affinity or unspecific binders. The remaining aptamers can be collected and propagated, e.g., PCR amplified, and used in subsequent rounds of selection. This selection process, referred to as Systemic Evolution of Ligands by Exponential Enrichment or SELEX, is commonly used for selecting and identifying highly targeted aptamers. A variant of this methodology, known as cell-SELEX, has been developed for aptamers that are capable of recognizing whole living cells.

### Aptamer Switch

In some embodiments, the antigen binding probe may comprise at least one of the four elements: a single-stranded oligonucleotide; a short, complementary DNA sequence to the oligonucleotide; a linking moiety that conjugates the oligonucleotide with the DNA sequence; and luminescent molecules.

In some embodiments, the single oligonucleotide has a first and second terminus, wherein the first terminus can be attached to a luminescent molecule and a second terminus attached to a piercing element. In some embodiments, the single oligonucleotide further comprises a short, partially complementary DNA sequence, wherein the short, partially complementary DNA sequence further comprises a second luminescent molecule.

In some embodiments, the single-stranded oligonucleotide has a first and second terminus, where the first terminus can be attached to the linking moiety. The linking moiety can be also attached to the first terminus of a short DNA strand having a partially complementary sequence to the oligonucleotide, where the short DNA strand has a first and second terminus. Luminescent molecules are attached to the second termini of the oligonucleotide and the short DNA strand.

In some embodiments, the oligonucleotide can be an aptamer. Aptamers are nucleic acid molecules that bind to a specific target molecule such as small molecules, proteins, nucleic acids, cells, tissues, and organisms. In some embodiments, aptamers are single-stranded oligonucleotides exhibiting high affinity and specificity toward any given target molecule. The aptamer disclosed herein may be any suitable size.

In some embodiments, the size of the aptamer as disclosed herein can be about 5 nucleotides to about 250 nucleotides. In some embodiments, the probe may further comprise a short, complementary DNA sequence that, in the absence of a target analyte, can hybridize with complete or partial complementary to a portion of the aptamer. In some embodiments, the aptamer and complementary DNA sequence described herein may have one or more mismatched nucleotides.

### Linking Moieties

In some embodiments the analyte sensor (e.g., analyte binding probe) may further comprise a flexible linker region that attaches the oligonucleotides (e.g., aptamer) to the short DNA sequence. In some cases, the linker moiety can be a nucleotide acid moiety that does not bind to either the oligonucleotide, the short DNA sequence, a peptide nucleic acid (PNA) moiety, a peptide moiety, a disulfide bond, a phosphodiester linkage, or a polymer such as a polyethylene glycol (PEG) moiety. The linker region disclosed herein can be about 2 residues in length to about 45 residues in length. In some embodiments, a linker can be a homopolymeric polynucleotide. An intramolecular linker can be about 5 nucleotides to about 60 nucleotides.

### Antigen Binding Probes: Peptides

In some embodiments, an analyte sensor (e.g., analyte binding probe) may be a polypeptide-based probe employing intramolecular signal transduction. In some embodiments, the polypeptide-based probe contains at least one of the following elements: a polypeptide with an antigen binding region, a linking moiety, and a luminescent molecule or a redox reporter. In some embodiments, the polypeptide-based probe is a protein-based affinity reagent. In some embodiments, the polypeptide-based probe comprises nanobodies, antibody fragments, peptides, cysteine-knot proteins (knottins), or combinations thereof.

In some embodiments, a polypeptide with an antigen binding domain is further conjugated with a luminescent molecule. In some embodiments, a polypeptide with an antigen binding domain is attached to a linking moiety. The linking moiety can be also attached to a second polypeptide with another antigen binding domain, and the luminescent molecules are attached to the first and second polypeptides. In some cases, when the two antigen binding domains bind to the molecular target, they become closer in proximity and can alter the optical signal via a fluorophore/quencher or FRET-type interaction to produce an optical signal.

Antibody fragments which recognize specific epitopes can be generated by known techniques. Antibody fragments are antigen binding portions of an antibody, such as F(ab′)2, Fab′, F(ab)2, Fab, Fv, scFv and the like. F(ab′)2 fragments can be produced by pepsin digestion of the antibody molecule and Fab′ fragments can be generated by reducing disulfide bridges of the F(ab′) 2 fragments. Alternatively, Fab′ expression libraries can be constructed (Huse et al., 1989, Science, 246:1274-1281) to allow rapid and easy identification of monoclonal Fab′ fragments with the desired specificity. F(ab)2 fragments may be generated by papain digestion of an antibody.

In some embodiments, the polypeptide is a single-chain polypeptide. In some embodiments of any of the single-chain polypeptides described herein, the single-chain polypeptide can be or include a BiTe, a (scFv)2, a nanobody, a nanobody-HSA, a DART, a TandAb, a scDiabody, a scDiabody-CH3, scFv-CH-CL-scFv, a HSAbody, scDiabody-HSA, or a tandem-scFv.

In some embodiments, the polypeptide is a multi-chain polypeptide. In some embodiments, the multi-chain polypeptide can be or can include an antibody, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIHI IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, an Intrabody, a dock and lock, a 1 mmTAC, an IgG-IgG conjugate, a Cov-X-Body, or a scFv1-PEG-scFv2.

In some embodiments, the antigen-binding domain is humanized or human.

The antibodies of use can be of various isotypes, such as human IgG1, IgG2, IgG3, or IgG4. The antibodies or fragments thereof can be chimeric human-mouse, humanized (human framework and murine hypervariable (CDR) regions), or fully human, as well as variations thereof, such as half-IgG4 antibodies (referred to as “unibodies”). The antibodies or fragments thereof may be designed or selected to comprise human constant region sequences that belong to specific allotypes, which may result in reduced immunogenicity when administered to a human subject. Preferred allotypes for administration include a non-G1m1 allotype (nGim1), such as Gim3, Gim3,1, Gim3,2, or Gim3,1,2. More preferably, the allotype is selected from the group consisting of the nG1m1, Gim3, nG1 m1,2, and Km3 allotypes.

In some embodiments, non-limiting examples of analyte binding probe includes a DARPin, an affibody, a monobody, a nanobody, a diabody, an antibody (including a monospecific or bispecific antibody); a cell-targeting oligopeptide including but not limited to RGD integrin-binding peptides, de novo designed binders, a bicycle peptide, conotoxins, small molecules such as folic acid, and a virus that binds to the cell surface.

### Antibody-Bait Switches

In some embodiments, the analyte sensor (e.g., analyte binding probe) is a single antibody construct comprising: an antibody or a binding fragment thereof comprising a first label, wherein the antibody or the binding fragment thereof is linked to a blocking analyte and a second label via a linker, wherein: in the absence of a target analyte, the blocking analyte binds to the antibody or the binding fragment thereof, and in the presence of the target analyte, the target analyte competes with the blocking analyte for binding to the antibody or the binding fragment thereof, and the first and second labels interact to generate a detectable readout that changes depending on whether the blocking analyte is bound by the antibody or the binding fragment thereof, or is not bound by the antibody or the binding fragment thereof. In some embodiments, the second label is conjugated to the linker. In some embodiments, the second label is conjugated to the blocking analyte. In some embodiments, the first label is a fluorophore and the second label is a quencher. In some embodiments, the first label is a quencher and the second label is a fluorophore. In some embodiments, the first label is a donor fluorophore and the second label is an acceptor fluorophore. In some embodiments, the first label is an acceptor fluorophore and the second label is a donor fluorophore. In some embodiments, the detectable readout is an optical signal, an electrical signal, an electrochemical signal, a nuclear magnetic resonance signal, or a biological signal. In some embodiments, the optical signal is a fluorescent signal. In some embodiments, binding of the target analyte to the antibody or the binding fragment thereof increases or decreases the detectable readout.

In some embodiments, the analyte sensor (e.g., analyte binding probe) is a single antibody construct comprising: an antibody or a binding fragment thereof comprising a redox reporter, wherein the antibody or the binding fragment thereof is linked to a blocking analyte and a sensing electrode; wherein: in the absence of a target analyte, the blocking analyte binds to the antibody or the binding fragment thereof, and in the presence of the target analyte, the target analyte competes with the blocking analyte for binding to the antibody or the binding fragment thereof, and the redox reporter and the sensing electrode interact to generate an electrical signal that changes depending on whether the blocking analyte is bound by the antibody or the binding fragment thereof, or is not bound by the antibody or the binding fragment thereof. In some embodiments, the antibody or the binding fragment thereof is linked to the blocking analyte and the sensing electrode via a linker. In some embodiments, the antibody or the binding fragment thereof is linked to the blocking analyte by a first linker and the antibody or the binding fragment thereof is linked to the sensing electrode via a second linker. In some embodiments, the antibody or the binding fragment thereof is linked to the sensing electrode via a gold-thiol bond. In some embodiments, the sensing electrode is a gold electrode.

### Dual-Antibody Switches

In some embodiments the analyte sensor (e.g., analyte binding probe) is a dual antibody construct comprising: (a) two detecting strands, wherein a first detecting strand comprises a first antibody or a binding fragment thereof, and a second detecting strand comprises a second antibody or a binding fragment thereof; and (b) a first label and a second label, wherein the detecting strands or portions thereof are complementary and hybridize to each other or one or more scaffold strands, and wherein in the presence of the target analyte, the first antibody and the second antibody bind to two different epitopes on the target analyte, and the first and second labels interact with each other to generate a detectable readout compared to when there is an absence of the target analyte.

In some embodiments, the two detecting strands hybridize to a scaffold strand, thereby linking the two detecting strands. In some embodiments, the two detecting strands hybridize to a single scaffold strand. In some embodiments, two detecting strands hybridize to separate scaffold strands which hybridize to each other. In some embodiments, the two detecting strands hybridize to each other, thereby linking the two detecting strands. In some embodiments, the first label is linked to the first antibody and the second label is linked to the second antibody. In some embodiments, the first label and/or second label is linked to a scaffold strand. In some embodiments, the first label is linked to a first label oligonucleotide that is hybridized to a scaffold strand and second label is linked to a second label oligonucleotide that is hybridized to a scaffold strand. In some embodiments, the first label is linked to a first scaffold strand and the second label is linked to a second scaffold strand. In some embodiments, in the presence of the target analyte, a portion of the first scaffold strand and a portion of the second scaffold strand hybridize to each other.

In some embodiments, the antibody construct generates fluorescence as a detectable readout when it binds to a target analyte. In some embodiments, one of the first and second labels on the antibody construct can be a fluorophore and the other of the first and second labels can be a quencher. In this case, the blocking analyte binds to the antibody in the absence of the target analyte and the fluorescence signal is quenched. In some embodiments, one of the first and second labels on the antibody construct can be a donor fluorophore and the other of the first and second labels can be an acceptor fluorophore and the first and second labels can form a FRET pair. In some embodiments, when the antibody is bound by the blocking analyte in the absence of a target analyte, the first and second labels are in proximity of each other to produce a FRET signal. The disappearance or reduction of the FRET fluorescence can serve as a signal of target analyte binding. In other words, in the absence of the target analyte, the fluorescent signal from the acceptor fluorophore can serve as the detectable signal. In the presence of the target analyte and the formation of the antibody-target analyte complex, the donor fluorophore and the acceptor fluorophore are not in proximity of each other to produce a FRET signal and the fluorescent signal of the donor fluorophore can serve as the detectable readout.

### Antibody-Aptamer Switches

In certain embodiments, the analyte sensors (e.g., analyte binding probes) comprise a capture agent that binds to a first epitope of an analyte, and an aptamer that binds to a second epitope of the analyte, where the aptamer is stably associated with the capture agent and produces a detectable signal upon cooperative binding of the capture agent and aptamer to the analyte. The capture agent mediates initial analyte binding due to its higher affinity, thereby increasing the local analyte concentration and leading to cooperative binding and signaling by the aptamer, e.g., a strand-displacement (SD) aptamer switch. In certain embodiments this design leads to an enhancement in sensitivity as compared to the aptamer alone, and the molecular sensor may exhibit reversible binding, enabling repeated measurements and retaining remarkable sensitivity even in interferent-rich samples.

A variety of suitable capture agents may be employed, non-limiting examples of which include a receptor (e.g., when the analyte is a ligand of the receptor), a ligand (e.g., when the analyte is a receptor for the ligand), a small molecule, an antibody, a T-cell receptor (TCR), etc. In some instances, the capture agent is an antibody. By “antibody” is meant an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the analyte. In certain embodiments, the antibody is an IgG, Fab, scFv, scFv-Fc, scFv-CH3, scFv-zipper, scFab, VHH/VH, or diabody.

In some embodiments, the detectable signal is an optically-detectable signal. For example, the detectable signal may be a fluorescent signal, e.g., where the aptamer or a displacement strand (if present) is labeled with a fluorophore. Non-limiting examples of approaches for the production of fluorescent signals upon cooperative binding of the capture agent and aptamer to the analyte include fluorophore-quencher interactions, Förster resonance energy transfer (FRET), and the like. In certain embodiments, the aptamer comprises a quencher moiety that quenches the fluorophore in a first conformation when the molecular sensor is not bound to the analyte, and upon cooperative binding of the capture agent and aptamer to the analyte, the aptamer assumes a second confirmation in which the fluorophore assumes an unquenched state.

In some embodiments, the aptamer is stably associated with the capture agent. As used herein, “stably associated” means a physical association between two entities in which the mean half-life of 25 association is one day or more in PBS at 4° C. In some embodiments, the physical association between the two entities has a mean half-life of one day or more, one week or more, one month or more, including six months or more, e.g., 1 year or more, in PBS at 4° C. According to some embodiments, the stable association arises from a covalent bond between the two entities, a non-covalent bond between the two entities (e.g., an ionic or metallic bond), or other forms of chemical attraction, such as hydrogen bonding, Van der Waals forces, and the like. The linkage between the aptamer and capture agent (e.g., antibody) can take the form of either direct conjugation of the aptamer to the capture agent, or hybridization of part of the aptamer sequence to an anchor DNA sequence conjugated to the capture agent. As such, the linker region may be composed of a combination of regions of the anchor strand and the aptamer strand.

### Analyte Detection

In some embodiments, the analyte binding domain of the analyte sensor (e.g., analyte binding probe) can change its conformation (e.g., Aptamer Switch) upon binding to a target analyte. In some embodiments, an analyte sensor (e.g., analyte binding probe) may be an antibody switch sensor or any other sensor that may undergo conformational rearrangement upon interaction with one or more analytes that modulates the optical signal. Suitable optical signals which can be used as an assay readout include any optical signal which can be generated by a proximity assay, such as those generated by fluorescence resonance energy transfer (FRET), fluorescence polarization, fluorescence quenching, phosphorescence technique, luminescence enhancement, luminescence quenching, diffraction or plasmon resonance, all of which are known techniques.

For example, in the absence of a target molecule, the analyte detection is in a position such that a first label (e.g., fluorophore) is in close proximity to a second label (e.g., quencher), the detectable optical read-out is quenched (e.g., reduced) by the second label. Conversely, when the analyte binding domain binds to its target molecule, the binding between binding domain and target molecule induces a conformational change, such that the first label (e.g., fluorophore) is away from the second label (e.g., quencher) resulting in the increase of detectable optical read-out.

Efficient and complete quenching of the fluorescence emitted from the fluorophore by the quencher depends in part on the overlap between the fluorophore emission and quencher absorption spectra. For example, the fluorophore coumarin emits at emission wavelength around 472 nm and can be paired with quencher (QSY35) which absorbs at a wavelength of around 475 nm. In another example, fluorophore Alexa 532 emits at emission wavelength around 554 nm and can be paired with quencher QSY7 which absorbs at wavelength around 560 nm. In yet another example, fluorophore Alex 647 emits at an emission wavelength around 665 nm and can be paired with quencher QSY 21 which absorbs at wavelength around 661 nm.

In some embodiments, the substrate incorporates assay components that generate an optical readout using FRET. In this assay format, a pair of fluorophores are used wherein one serves as a donor chromophore and the other acts as an acceptor chromophore. With respect to the fluorescence emission spectrum, the emission spectrum of the donor chromophore overlaps with the absorption spectrum of the acceptor chromophore, such that when the donor and acceptor chromophores are brought into close proximity, a proportion of the energy which normally would produce fluorescence emitted by the donor chromophore (following irradiation with incident radiation of a wavelength absorbed by the donor chromophore) will be non-radiatively transferred to the adjacent acceptor chromophore, a process known in the art as fluorescence resonance energy transfer, with the result that a proportion of the fluorescent signal emitted by the donor chromophore is quenched, that the lifetime of the fluorescence is changed, and, in some instances, that the acceptor chromophore emits fluorescence. The acceptor chromophore may, however, be a non-fluorescent dye. Fluorescence resonance energy transfer generally only occurs when the donor and acceptor chromophores are brought into close proximity by the binding of, for instance, an analyte to an aptamer, which causes a conformational change which brings the donor and acceptor chromophores together. Thus, in the presence of analyte, the amount of quenching can be increased (resulting in a measurable decrease in the intensity of the fluorescent signal emitted by the donor chromophore or an increase in the intensity of the signal emitted by the acceptor chromophore). The intensity or lifetime of the fluorescent signal emitted from the donor chromophore thus correlates with the concentration of target analyte in the interstitial fluid bathing the sensor.

The sensor can be adapted for the detection or quantitative measurement of any target analyte present in interstitial fluid such as glucose (in connection with the long-term monitoring of diabetics), urea (in connection with kidney disease or dysfunction), lactate (in connection with assessment of muscle performance in sports medicine), ions such as sodium, calcium or potassium and therapeutic drugs whose concentration in the blood must be closely monitored, such as, for example, digoxin, theophylline or immunosuppressant drugs. The above analytes are listed by way of example only and it is to be understood that the precise nature of the analyte to be measured is not material.

The sensor can be interrogated transcutaneously using optical means, for example, no physical connection may be required between the sensor and the optical means. When the sensor incorporates the technique of fluorescence resonance energy transfer, the optical means can supply a first beam of incident radiation at a wavelength within the absorption spectrum of the donor chromophore and a second beam of incident radiation at a wavelength within the absorption spectrum of the acceptor chromophore. In addition, the optical means can be capable of measuring optical signals generated in the sensor at two different wavelengths; wavelength 1 within the emission spectrum of the donor chromophore (the signal generated in connection with the measurement of analyte and wavelength 2 in the emission spectrum of the acceptor chromophore (which could be the analyte signal or the internal reference or calibration signal).

Optical means suitable for use in remote interrogation of the sensor can include a simple high-throughput fluorimeter comprising an excitation light source such as, for example, a light-emitting diode (for example blue, green or red), an excitation light filter (for example a dichroic, dye filter) and a fluorescent light detector for example (PIN diode, Silicon photo-multiplier, Avalanche photodiode, image sensor configuration).

### Electrochemical Detection

In some embodiments the analyte binding probes can be configured as electrochemical sensors. For example, an aptamer switch can be immobilized on an electrode (e.g., gold electrode) and labeled with a redox reporter, such as methylene blue. Upon analyte binding, the analyte binding probes undergo a change in conformational state which causes the distance between the redox reporter and the surface of the electrode to change. This change in distance causes a change in electrical signal in response to the target analyte which can be used to measure the analyte concentration.

### Enzymes Sensors

Although the preferred embodiment of the device leverages affinity-based analyte binding probes, more conventional enzymatic approaches can also be used in the device. The enzymatic approaches can be used either alone, or in combination, with the other analyte binding probes and signaling mechanisms described in this application.

### EXAMPLES

### Example 1: Real-Time Cortisol Measurement Using Benchtop Reader

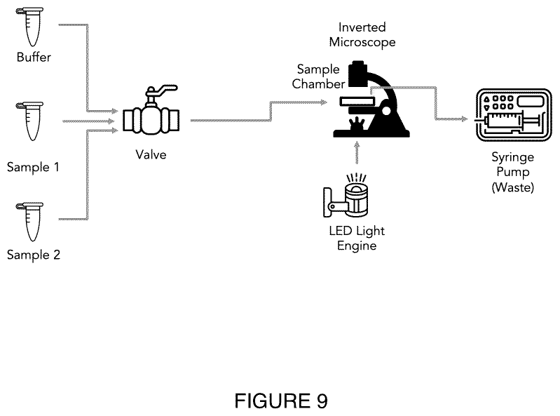

Described below is an example device for continuously measuring cortisol in sample solutions on the benchtop. The benchtop reader (FIG. 9) moves samples through a microfluidic chip for detection on a fluorescence microscope. The device can be used for the various applications, use cases, and indications described throughout this application.

In this example, 5 mL samples of known or unknown concentrations of cortisol are connected to a programmable motorized 10-to-1 valve. The inlet of each section of tubing is submerged directly in the corresponding sample solution. The outlet of each section of tubing couples to one of the inlets of the valve via a ¼″-28 threaded, PFA flangeless fitting and ETFE ferrule.

The motor of the valve rotates to connect one of the 10 inlets to its 1 outlet via an internal fluidic path. Which inlet is selected is controlled by a computer running a python script communicating through one of its USB ports.

The outlet of the valve leads to the inlet of a microfluidic chip. These are connected via a 25 Gauge steel pin pressed into a silicone connector adhered to the top of the chip. The chip consists of a clear polycarbonate cover adhered to a glass slide. The chamber of this chip is approximately 50 μL in volume and has one inlet and one outlet that are 1.5 mm in diameter. The entire chip is approximately 1″ in width, 3″ in length, and .1″ in height. Inside the chamber are analyte binding probes, which are conjugated to magnetic particles. The magnetic particles are made of magnetic polymer matrix and have a mean diameter of 3 μm. The beads are immobilized in the chamber using a permanent magnet adhered to the top of the microfluidic chip. In this embodiment, the analyte binding probes are aptamer switches.

In this example, the outlet of the microfluidic chip is attached to a 60 mL syringe. These are connected with another section of PTFE tubing that is 1/16″ in outer diameter and .5 mm in inner diameter. The tubing connects to the outlet of the chip via a 25 Gauge steel pin pressed into a silicone connector adhered to the chip. The tubing is attached to the syringe by slipping the tubing over a 25 Gauge blunt tip needle, coupled to the syringe via a luer lock. The 60 mL syringe is affixed to a syringe pump. The syringe pump is programmable and can pump and withdraw at rates ranging from approximately 29 μL/hr to 35 mL/min with a 60 ml syringe. Lower flow rates can be achieved with smaller syringes, but in this embodiment, the pump withdraws fluid at a rate of approximately 333 μL/min, driving the fluid through the system from the 5 mL samples all the way to the syringe. The pump can be started and stopped by the same python script that controls the valve, coordinating their actions. The pump is used to exchange the fluid in the microfluidic chamber every 10 minutes, giving time for the aptamer switches on magnetic beads to equilibrate before each exchange. The pump flows 500 μL per exchange to ensure there is no residual solution from before the exchange left in the chamber.

In this example, the microfluidic chip is affixed to the stage of an inverted fluorescence microscope. The microscope delivers red light with peak wavelength of 637 nm to excite the luminescent molecules within the aptamer switches. The emission light of the luminescent molecules passes through the optical stack of the microscope, including a cy5 filter cube, and is quantified by a camera attached to the microscope. The images are taken using a 20× objective and 250 ms exposure time. In this embodiment, images are taken automatically on the microscope every 2 minutes and are saved to a computer.

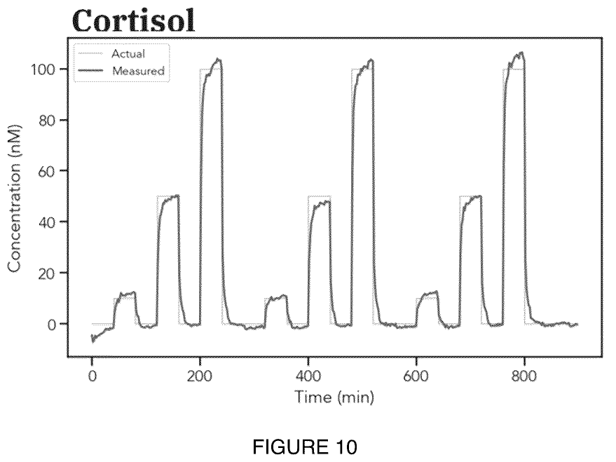

The images stored on the computer can be used to quantify the signal of the aptamer switches on magnetic beads over time. Measured brightness values can be mapped to concentrations using a calibration curve generated from running samples of known concentrations through the benchtop device. The resulting concentration measurements can be plotted over time (FIG. 10).

### Example 2: Wearable Device for Periodic Cortisol Measurement in Dermal ISF

Described below is an example device for continuously measuring cortisol in the dermal ISF of a subject. The device can be used for the various applications, use cases, and indications described throughout this application.

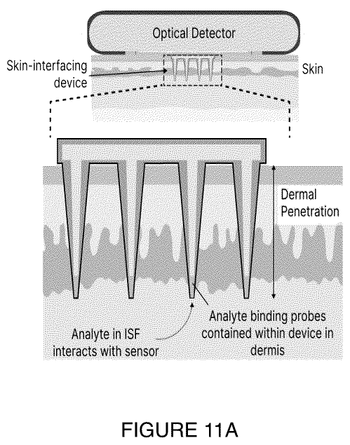

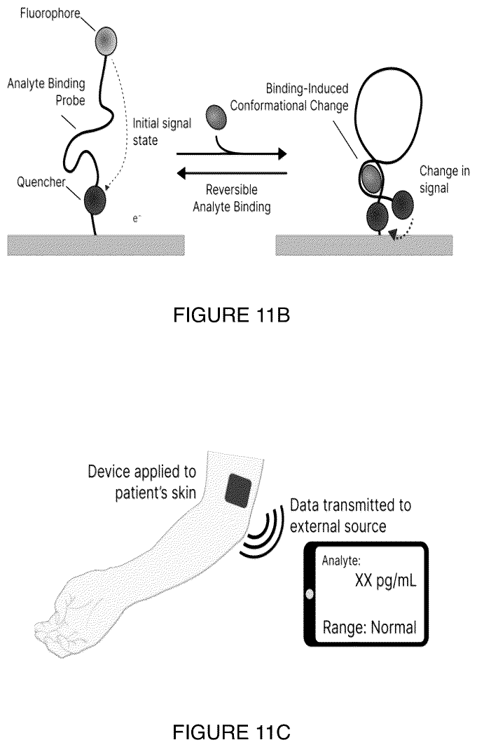

The body-worn device (examples shown in FIG. 11A, FIG. 11B, and FIG. 11C) can be cubic in shape and measures approximately 25×25×10 millimeters. The body-worn device consists of two components—a reusable optoelectronics reader and a disposable microneedle array patch (MAP) biosensor patch. The two device parts can be locked together, and then the microneedles are inserted into the dermis of the subject. After 10-14 days, the disposable patch is discarded and then re-usable reader is re-charged prior to re-use with a new patch.

The device measures analyte sensing by using molecule-specific optical molecular switches contained within a disposable hollow microneedle array patch (MAP) that can be inserted painlessly into the patient's skin to sample the biomarker content of dermal ISF. In this example, the MAP consists of six 30 gauge stainless steel metal microneedles measuring 1.5 millimeters in depth. Three of the microneedles in the MAP contain analyte binding probes for cortisol, while the other three contain analyte binding probes for DHEA-S. Once inserted into the subject, analytes from the dermal ISF can diffuse and interact with the aptamer switches contained within the MAP biosensor patch. The aptamer switches are covalently attached to polystyrene beads which are physically entrapped within a PEGDMA-based hydrogel matrix contained within the hollow microneedles.

The optoelectronic reader is configured to measure fluorescent signal from the aptamer switches by delivering excitation light at 632 nanometers into the microneedles and then collecting emission light at a larger wavelength from the fluorophores contained within the aptamer switches. The excitation light is generated from a vertical-cavity surface-emitting laser. The emission light from the aptamer switches is filtered using a dichroic filter from the emission light and then measured using a CMOS image sensor. By measuring the fluorescent intensity of the aptamer switches over time, the concentration of the analyte can be measured periodically. In this case, the device contains analyte binding probes for cortisol and DHEA-S and can quantify the concentration over time of both molecules. The device is configured to conduct a measurement every 5-15 minutes. This duty cycling of the device saves energy to extend the lifetime that the device can be worn prior to re-charging.

The device transmits the cortisol and DHEA-S levels remotely from the sensor to another device (e.g., cell phone) or to the cloud. The features of the cortisol profile (which can optionally use DHEA-S to provide a stable reference) are then extracted. The analyte concentration profiles, alerts, or guidance that can then be transmitted to a user or a healthcare professional.

U.S. Pat. No. 12,109,019 issued Oct. 8, 2024 titled Systems and Methods for Analyte Detection is hereby incorporated by reference in its entirety and provides a readily applicable optical electronic MAP based detection system and detailed description with accompanying figures of an optical system in the form of a disposable at least partially hollow microneedle array where each needle is filled with a molecular sensing domain including analyte binding probes that undergo a change in signal upon interacting with an analyte and these changes are detected by the optical detector for the optical detection and measurement of a biomarker and its concentration.

However, the system can be otherwise configured.

### 5. Specific Examples

A list of specific examples of the technology described herein are provided below. A person of skill in the art will recognize that the scope of the technology is not limited to and/or by these specific examples.
Specific Example 1. A method, comprising: using a wearable system, determining a time series of cortisol levels in an interstitial fluid of a user, the time series of cortisol levels corresponding to a time period of at least 6 hours, the wearable system comprising a piercing element, an analyte binding probe, and a reader, wherein the piercing element is configured to pierce a body surface of a user, thereby bringing the piercing element in contact with the interstitial fluid of the user, wherein determining each cortisol level in the time series comprises: using the reader, recording a signal emitted by the analyte binding probe, wherein the analyte binding probe comprises an aptamer in contact with analytes from the interstitial fluid, wherein the aptamer is configured to bind to cortisol and is configured to change in conformation in response to binding to cortisol, and determining the cortisol level based on the recorded signal; extracting a set of features from the time series of cortisol levels; and characterizing a health metric of the user based on the set of features.
Specific Example 2. The method of Specific Example 1, wherein the reader comprises an optoelectronic reader, wherein the signal emitted by the analyte binding probe comprises light.
Specific Example 3. The method of any of Specific Examples 1-2, wherein the wearable system further comprises a second analyte binding probe, wherein determining each cortisol level in the time series of cortisol levels further comprises: using the reader, recording a second signal emitted by the second analyte binding probe, wherein the second analyte binding probe comprises a second aptamer in contact with the analytes from the interstitial fluid, the second aptamer having a different nucleotide sequence than the aptamer, wherein the second aptamer is configured to bind to cortisol and is configured to change in conformation in response to binding to cortisol, wherein the cortisol level is further based on the second signal.
Specific Example 4. The method of Specific Example 3, wherein the aptamer is configured to bind to cortisol with a first binding affinity and configured to bind to a secondary analyte with a second binding affinity, wherein the second aptamer is configured to bind to cortisol with a third binding affinity and configured to bind to the secondary analyte with a fourth binding affinity.
Specific Example 5. The method of any of Specific Examples 1-4, further comprising: using the wearable system, determining a time series of levels of a second analyte in the interstitial fluid of the user, the time series of levels of a second analyte corresponding to the time period, the wearable system further comprising a second analyte binding probe, wherein determining each level of the second analyte in the time series comprises: using the reader, recording a second signal emitted by the second analyte binding probe, wherein the second analyte binding probe comprises a second aptamer in contact with the analytes from the interstitial fluid, wherein the second aptamer is configured to bind to cortisol and is configured to change in conformation in response to binding to cortisol, and determining the level of the second analyte based on the second signal; and extracting a second set of features from the time series of levels of the second analyte, wherein the health metric of the user is further characterized based on the second set of features.
Specific Example 6. The method of Specific Example 5, wherein the second aptamer comprises an analyte binder to at least one of: glucose, glucocorticoid, cortisone, corticosterone prednisolone, prednisone, methylprednisolone, DHEA, DHEA-S, insulin, glucagon, estrogen, progesterone, or testosterone.
Specific Example 7. The method of any of Specific Examples 5-6, wherein the second aptamer comprises an analyte binder to dexamethasone, and wherein the health metric comprises at least one of: a confirmation that the user has taken dexamethasone, a dexamethasone suppression test evaluation score, a characterization of dexamethasone metabolism of the user, or a characterization of dexamethasone suppression of cortisol.
Specific Example 8. The method of any of Specific Examples 1-7, further comprising: measuring a temperature; and based on the measured temperature, applying a temperature correction to the recorded signal to produce a temperature-corrected signal, wherein the cortisol level is determined based on the temperature-corrected signal.
Specific Example 9. The method of any of Specific Examples 1-8, further comprising: applying a temperature correction to the recorded signal to produce a temperature-corrected signal, wherein the cortisol level is determined based on the temperature-corrected signal, wherein the temperature correction is configured to correct for temperature-dependent changes in a characteristic of the analyte binding probe, the characteristic of the analyte binding probe characteristic comprising at least one of: baseline signal, signal gain, signal dynamic range, binding affinity, binding sensitivity, binding kinetics, or redox reporter dynamics.
Specific Example 10. The method of any of Specific Examples 1-9, further comprising: applying a temperature correction to the recorded signal to produce a temperature-corrected signal, wherein the cortisol level is determined based on the temperature-corrected signal, wherein the temperature correction is configured to correct for temperature dependence of at least one of: fluorophore brightness, quenching, lifetime, excitation source output, reader response, electronic drift, electron transfer kinetics, or square wave frequency dependence.
Specific Example 11. The method of any of Specific Examples 1-10, wherein the health metric is used to aid in the diagnosis, monitoring, and/or treatment of a condition associated with cortisol, the method further comprising transmitting the health metric to a physician.
Specific Example 12. The method of Specific Example 11, wherein the condition comprises at least one of: adrenal insufficiency, Cushing disease, Cushing syndrome, diabetes, hypertension, obesity, osteoporosis, mild autonomous cortisol secretion (MACS), hypercortisolemia, a sleep disorder, a mental disorder, long COVID, adrenal tumors, Cyclical Cushing syndrome, hypercortisolism, Addison's disease, glucocorticoid withdrawal syndrome, parathyroid dysfunction, or an autoimmune condition.
Specific Example 13. The method of any of Specific Examples 1-12, further comprising adjusting an administration of a drug to the user based on the health metric, wherein adjusting the administration of the drug comprises adjusting at least one of: a timing of dosing, a dosage, a number of doses per day, or a tapering protocol.
Specific Example 14. The method of Specific Example 13, wherein the drug comprises at least one of: adrenal steroidogenesis inhibitors, glucocorticoid, or glucocorticoid receptor blockers.
Specific Example 15. The method of any of Specific Examples 1-14, wherein the health metric is used to monitor at least one of: pain, inflammation, cognitive function, stress, athletic recovery, surgery recovery, fertility, metabolic health, menstruation, or diurnal cortisol rhythm
Specific Example 16. The method of any of Specific Examples 1-15, wherein the health metric is further determined based on biomarker data received from a wearable sensor, wherein the biomarker data comprises at least one of: heart rate, heart rate variability, respiration rate, sleep tracking data, photoplethysmography data, accelerometer data, or glucose data.
Specific Example 17. The method of any of Specific Examples 1-16, wherein the health metric is further determined based on patient-reported data received from the user.
Specific Example 18. The method of any of Specific Examples 1-17, wherein the analyte binding probe comprises a reporter molecule, wherein the signal emitted by the analyte binding probe is emitted by the reporter molecule.
Specific Example 19. The method of any of Specific Examples 1-18, wherein the set of features comprises at least one of: mean, daily maximum, daily minimum, a rate of change, a time at which the daily minimum occurs, a time at which the daily maximum occurs, a time at which a cortisol level increase occurs, an area under the time series of cortisol levels, or a curve shape.
Specific Example 20. The method of any of Specific Examples 1-19, wherein the time series of cortisol levels comprises at least one cortisol level every 5 minutes.

In variants, the present disclosure provides: A method of measuring an analyte in a human using a body-worn device configured to periodically measure the levels of an analyte (total or free) from a physiological fluid of a wearer, where the analyte is measured at least once in a 24 hr period obtained from the body-worn device in order to extract features of the subject's analyte rhythm, and using said features to aid in the diagnosis, monitoring, and/or treatment of conditions impacted by analyte levels.

In a specific example, the analyte is a glucocorticoid.

In a specific example, the glucocorticoid is cortisol.

In a specific example, the glucocorticoid is cortisone.

In a specific example, the glucocorticoid is prednisolone.

In a specific example, the glucocorticoid is prednisone.

In a specific example, the physiological fluid is interstitial fluid.

In a specific example, the conditions impacted by cortisol levels include but are not limited to Cushing disease, Cushing syndrome, mild autonomous cortisol secretion (MACS), Adrenal Insufficiency, Glucocorticoid Drug Dosing, stress, fertility, athletic performance, diabetes, hypertension, obesity, or osteoporosis.

In a specific example, a feature is the mean cortisol level throughout a 24 hour period.

In a specific example, a feature is the mean cortisol level throughout a period longer than 1 hour but shorter than 24 hours.

In a specific example, a feature is the mean cortisol level throughout a period longer than 5 minutes but shorter than 1 hour.

In a specific example, a feature is the mean cortisol through a period longer than 24 hours but shorter than or equal to 2 weeks.

In a specific example, a feature is a maximum cortisol level during a 24 hour period.

In a specific example, a feature is a minimum cortisol level during a 24 hour period.

In a specific example, a feature is the difference between a daily maximum and daily minimum cortisol measurement.

In a specific example, a feature is the time at which a daily peak (i.e. daily maximum cortisol level) cortisol level occurs.

In a specific example, a feature is the time at which a daily trough (i.e. daily minimum) cortisol level occurs.

In a specific example, a feature is the start time of an increase in cortisol secretion.

In a specific example, the start time of an increase in cortisol secretion is marked by an increase in the rate of change above a certain threshold after a certain time of day.

In a specific example, the start time of an increase in cortisol secretion is marked by the cortisol level crossing a threshold at a defined percentage of the daily maximum cortisol peak.

In a specific example, the start time of an increase in cortisol secretion is marked by the cortisol level crossing a threshold at a defined percentage of the difference between daily maximum cortisol peak and daily minimum cortisol trough.

In a specific example, a feature is the number of local cortisol maxima above a certain threshold from the surrounding local minima (i.e. cortisol spikes) occurring within a 24 hour period.

In a specific example, the cortisol signals are first low-pass filtered before extracting the number of local cortisol maxima.

In a specific example, a feature is the maximum cortisol level occurring within 2-hours after a meal.

In a specific example, a feature is the time between a daily peak and daily trough.

In a specific example, a feature is the cortisol awakening response (CAR)

In a specific example, the feature is the cortisol response within 1 hour of waking.

In a specific example, the feature is the output of a machine learning algorithm.

In a specific example, the feature is derived from a fourier analysis.

In a specific example, the feature is derived from principal component analysis (PCA).

In a specific example, a feature is the morning cortisol level after administration of a 1 mg dose of dexamethasone before bedtime.

In a specific example, a feature is the difference between the morning cortisol level on a day without dexamethasone administration and the morning cortisol level after a 1 mg dose of dexamethasone before bedtime.

In a specific example, the method is used to diagnose or treat Cushing disease.

In a specific example, the method is used to detect the presence of hypercortisolemia.

In a specific example, the method is used to evaluate the patient response to a dexamethasone suppression test.

In a specific example, the method is used to monitor the patient response to glucocorticoid receptor blockers.

In a specific example, the method is used to monitor the patient response to adrenal steroidogenesis inhibitors.

In a specific example, the method is used to adjust the dosage of adrenal steroidogenesis inhibitors to attain a target cortisol level.

In a specific example, the method is used to monitor the patient's recovery from pituitary surgery and assess probability of recurrence.

In a specific example, the method is used to monitor the cortisol level after surgery to determine if morning cortisol levels are below a threshold prognostic of remission.

In a specific example, the method is used to assess normalization of the patient's diurnal cortisol rhythm and adequate suppression of nighttime cortisol levels.

In a specific example, the method is used to diagnosis or treat mild autonomous cortisol secretion (MACS).

In a specific example, the method is applied longitudinally to monitor the progression of patient hypercortisolemia.

In a specific example, the method is used to adjust the administration of a glucocorticoid.

In a specific example, the glucocorticoid is prednisone, cortisone, hydrocortisone, dexamethasone, triamcinolone, methylprednisolone, or clobetasol propionate.

In a specific example, the timing of the glucocorticoid dosing is changed.

In a specific example, the dosage of the glucocorticoid is changed.

In a specific example, the number of doses per day of the glucocorticoid is changed.

In a specific example, the adjustment is made to achieve similarity to a typical healthy cortisol diurnal rhythm.

In a specific example, the method is used to adjust the tapering protocol for a glucocorticoid.

In a specific example, the method is used to monitor the patient's endogenous cortisol levels 24 hours after ceasing administration of a glucocorticoid drug.

In a specific example, the measured cortisol features are compared to healthy references to assess for glucocorticoid-induced adrenal insufficiency.

In a specific example, the method is used to diagnose or treat insomnia or other sleep disorders.

In a specific example, the method is used to diagnose or treat mental disorders such as depression, PTSD, bipolar disorder, or mental fatigue.

In a specific example, the method is used to monitor athletic recovery.

In a specific example, the method is used to increase fertility.

In a specific example, the method is used to improve metabolic health.

In a specific example, the method is used to diagnose or treat long covid.

In a specific example, the cortisol measurements and features are transmitted to a designated health care provider using a remote monitoring including a plurality of cloud-based notifications.

In a specific example, recommendations regarding the diagnosis, monitoring, or treatment of the subject are transmitted to a designated health care provider.

In a specific example, recommendations on daily activities such as sleeping, eating, exercise, and/or meditation made to a user in order to promote a desired cortisol rhythm.

In a specific example, for the device of above, wherein said sensor includes at least one of an electrochemical sensor, an optical sensor, a galvanic sensor, a voltammetric sensor, an amperometric sensor, a potentiometric sensor, an impedimetric sensor, a resistive sensor, a capacitive sensor, an ultrasonic sensor, a radio-frequency sensor, or a microwave sensor.

In a specific example, for the device of above, wherein said sensor is an enzymatic electrochemical sensor.

In a specific example, the enzyme is 11ß-Hydroxysteroid Dehydrogenase type 1.

In a specific example, the enzyme is 11ß-Hydroxysteroid Dehydrogenase type 2.

In a specific example, for the device of above, wherein said sensor is a microneedle-based sensor which is contacted with the dermal ISF of a subject and configured to measure the concentration of one or more analytes using optically-responsive aptamer switches.

In a specific example, for the device of above, wherein said sensor is a microneedle-based sensor which is contacted with the dermal ISF of a subject and configured to measure the concentration of one or more analytes using e-chem aptamer switches.

In a specific example, the analyte binding probe is an aptamer switch.

In a specific example, in the analyte binding probe is an antibody-bait switch.

In a specific example, the analyte binding probe is a dual-antibody switch.

In a specific example, the analyte binding probe is an antibody-aptamer switch.

In a specific example, for the device of above, wherein said physiological fluid includes at least one of blood, serum, plasma, interstitial fluid, dermal interstitial fluid, extracellular fluid, intracellular fluid, sweat, or cerebrospinal fluid.

In a specific example, for the device of above, wherein said wearable device includes at least one of a transdermal sensor, transcutaneous sensor, an intradermal sensor, an intracutaneous sensor, a subdermal sensor, or a subcutaneous sensor.

In a specific example, for the device of above, wherein said wearable device penetrates the dermis to make cortisol measurements in dermal interstitial fluid.

All references cited herein are incorporated by reference in their entirety, except to the extent that the incorporated material is inconsistent with the express disclosure herein, in which case the language in this disclosure controls.

As used herein, “substantially” or other words of approximation can be within a predetermined error threshold or tolerance of a metric, component, or other reference, and/or be otherwise interpreted.

Optional elements, which can be included in some variants but not others, are indicated in broken line in the figures.

Different subsystems and/or modules discussed above can be operated and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

1. A method, comprising:
using a wearable system, determining a time series of cortisol levels in an interstitial fluid of a user, the time series of cortisol levels corresponding to a time period of at least 6 hours, the wearable system comprising a piercing element, an analyte binding probe, and a reader, wherein the piercing element is configured to pierce a body surface of a user, thereby bringing the piercing element in contact with the interstitial fluid of the user, wherein determining each cortisol level in the time series comprises:
using the reader, recording a signal emitted by the analyte binding probe, wherein the analyte binding probe comprises an aptamer in contact with analytes from the interstitial fluid, wherein the aptamer is configured to bind to cortisol and is configured to change in conformation in response to binding to cortisol, and
determining the cortisol level based on the recorded signal;
using the wearable system, determining a time series of levels of a second analyte in the interstitial fluid of the user, the time series of levels of the second analyte corresponding to the time period, the wearable system further comprising a second analyte binding probe, wherein determining each level of the second analyte in the time series comprises:
using the reader, recording a second signal emitted by the second analyte binding probe, wherein the second analyte binding probe comprises a second aptamer in contact with the aptamers from the interstitial fluid, and
determining the level of the second analyte based on the second signal;
extracting a set of features from the time series of cortisol levels;
extracting a second set of features from the time series of levels of the second analyte, wherein the health metric of the user is further characterized based on the second set of features; and
characterizing a health metric of the user based on the set of features and the second set of features.

2. The method of claim 1, wherein the reader comprises an optoelectronic reader, wherein the signal emitted by the analyte binding probe comprises light.

3. The method of claim 1, the second aptamer has a different nucleotide sequence than the aptamer, wherein the second aptamer is configured to bind to cortisol and is configured to change in conformation in response to binding to cortisol, wherein the cortisol level is further based on the second signal.

4. The method of claim 3, wherein the aptamer is configured to bind to cortisol with a first binding affinity and configured to bind to a secondary analyte with a second binding affinity, wherein the second aptamer is configured to bind to cortisol with a third binding affinity and configured to bind to the secondary analyte with a fourth binding affinity.

5. (canceled)

6. The method of claim 1, wherein the second aptamer comprises an analyte binder to at least one of: glucose, glucocorticoid, cortisone, corticosterone prednisolone, prednisone, methylprednisolone, DHEA, DHEA-S, insulin, glucagon, estrogen, progesterone, or testosterone.

7. The method of claim 1, wherein the second aptamer comprises an analyte binder to dexamethasone, and wherein the health metric comprises at least one of: a confirmation that the user has taken dexamethasone, a dexamethasone suppression test evaluation score, a characterization of dexamethasone metabolism of the user, or a characterization of dexamethasone suppression of cortisol.

8. The method of claim 1, further comprising:
measuring a temperature; and
based on the measured temperature, applying a temperature correction to the recorded signal to produce a temperature-corrected signal, wherein the cortisol level is determined based on the temperature-corrected signal.

9. The method of claim 1, further comprising: applying a temperature correction to the recorded signal to produce a temperature-corrected signal, wherein the cortisol level is determined based on the temperature-corrected signal, wherein the temperature correction is configured to correct for temperature-dependent changes in a characteristic of the analyte binding probe, the characteristic of the analyte binding probe characteristic comprising at least one of: baseline signal, signal gain, signal dynamic range, binding affinity, binding sensitivity, binding kinetics, or redox reporter dynamics.

10. The method of claim 1, further comprising: applying a temperature correction to the recorded signal to produce a temperature-corrected signal, wherein the cortisol level is determined based on the temperature-corrected signal, wherein the temperature correction is configured to correct for temperature dependence of at least one of: fluorophore brightness, quenching, lifetime, excitation source output, reader response, electronic drift, electron transfer kinetics, or square wave frequency.

11. The method of claim 1, wherein the health metric is used to aid in the diagnosis, monitoring, and/or treatment of a condition associated with cortisol, the method further comprising transmitting the health metric to a physician.

12. The method of claim 11, wherein the condition comprises at least one of: adrenal insufficiency, Cushing disease, Cushing syndrome, diabetes, hypertension, obesity, osteoporosis, mild autonomous cortisol secretion (MACS), hypercortisolemia, a sleep disorder, a mental disorder, long COVID, adrenal tumors, Cyclical Cushing syndrome, hypercortisolism, Addison's disease, glucocorticoid withdrawal syndrome, parathyroid dysfunction, or an autoimmune condition.

13. The method of claim 1, further comprising adjusting an administration of a drug to the user based on the health metric, wherein adjusting the administration of the drug comprises adjusting at least one of: a timing of dosing, a dosage, a number of doses per day, or a tapering protocol.

14. The method of claim 13, wherein the drug comprises at least one of:
adrenal steroidogenesis inhibitors, glucocorticoid, or glucocorticoid receptor blockers.

15. The method of claim 1, wherein the health metric is used to monitor at least one of: pain, inflammation, cognitive function, stress, athletic recovery, surgery recovery, fertility, metabolic health, menstruation, or diurnal cortisol rhythm.

16. The method of claim 1, wherein the health metric is further determined based on biomarker data received from a wearable sensor, wherein the biomarker data comprises at least one of: heart rate, heart rate variability, respiration rate, sleep tracking data, photoplethysmography data, accelerometer data, or glucose data.

17. The method of claim 1, wherein the health metric is further determined based on patient-reported data received from the user.

18. The method of claim 1, wherein the analyte binding probe comprises a reporter molecule, wherein the signal emitted by the analyte binding probe is emitted by the reporter molecule.

19. The method of claim 1, wherein the set of features comprises at least one of: mean, daily maximum, daily minimum, a rate of change, a time at which the daily minimum occurs, a time at which the daily maximum occurs, a time at which a cortisol level increase occurs, an area under the time series of cortisol levels, or a curve shape.

20. The method of claim 1, wherein the time series of cortisol levels comprises at least one cortisol level every 5 minutes.
